(19) United States
(12) Patent Application Publication
(10) Pub. No.: US 2022/0074845 A1
(43) Pub. Date: Mar. 10, 2022

(54) SYSTEMS AND METHODS FOR ANALYZING A FLUID SAMPLE

(71) Applicant: Pixcell Medical Technologies Ltd., Yokneam IIit (IL)

(72) Inventors: Vladimir MUZYKOVSKI, Hazorea (IL); Barak MAROM, Kiryat Haim (IL); Avishay BRANSKY, Kyriat Tivon (IL)

(73) Assignee: Pixcell Medical Technologies Ltd., Yokneam IIit (IL)

(21) Appl. No.: 17/418,430

(22) PCT Filed: Jan. 2, 2020
(86) PCT No.: PCT/IB2020/000026

§ 371 Date: Jun. 25, 2021

Related U.S. Application Data
(60) Provisional application No. 62/787,967, filed on Jan. 3, 2019.

(51) Int. Cl. G01N 15/14; G01N 33/53

(52) U.S. Cl. CPC G01N 15/1463; G01N 15/147; G01N 33/5302; G01N 15/1459; G01N 15/1436

(57) ABSTRACT

The invention relates to systems and methods for performing one or more immunoassays on a fluid sample and performing image analysis on the fluid sample as the sample is flowing so as to obtain measurements related to one or more target analytes based on image analysis. The systems and methods include a disposable fluidic device, such as a cartridge, configured to be loaded with a fluid sample and perform one or more assays on the fluid sample. The cartridge is further configured to flow a volume of fluid sample, undergoing, or having undergone, an assay, through a portion thereof to be subsequently analyzed by an analysis instrument. The analysis instrument is configured to capture images of the fluid sample as it is flowing through a portion of the cartridge and subsequently analyze the images so as to obtain measurements of one or more target analytes within the fluid sample.

### CROSS-REFERENCE TO RELATED APPLICATION(S)

This application claims priority to, and the benefit of, U.S. Provisional Application No. 62/787,967, filed Jan. 3, 2019, the content of which is incorporated by reference herein in its entirety.

### FIELD OF THE INVENTION

The invention generally relates to fluid sample analysis systems and methods.

### BACKGROUND

Turbidimetry is the process of determining the amount of cloudiness, or turbidity, in a solution based upon measurements of light interaction with particles suspended within the solution. Turbidimetric assays are used to determine, among other things, a concentration of a target analyte within a fluid sample. Immunoturbidimetry and nephelometry are two common methods used in immunoassays to assess the concentration of a target analyte, such as a certain molecule (e.g., a protein) within a fluid sample. Such methods generally rely on an antigen-antibody reaction, in which micro- or nano-beads coated with an antibody specific to the molecule are suspended in a liquid reagent and mixed with the fluid sample. In the presence of the target molecule, the antibodies and the antigen cluster to form an immune complex such that aggregation of the beads occurs as the beads start coupling to each other through bridges created by conjugates. The dynamics of aggregation (e.g., the rate and size of aggregates) is indicative of the molecule concentration.

Current systems and methods assess aggregation dynamics via various light measurements. For example, when light is passed through a fluid sample post antigen-antibody reaction, some light is scattered by the bead aggregates, some light is absorbed by the bead aggregates, and remaining light passes through the fluid sample. Immunoturbidimetry measures the absorbance of the light by the bead aggregates sample, in which the molecule concentration may be inversely proportional to the transmitted light signal, while nephelometry measures the scattering of certain wavelengths, in which there is a correlation between wavelength scattering and the size of a particle or aggregate.

While current analysis instruments are available for measuring the concentration of certain proteins in bodily fluids based on turbidimetric assays, such analysis instruments suffer from several drawbacks. For example, while current analysis instruments may be able to measure protein concentration in a sample, such measurements are limited. In particular, current instruments provide a single signal representing the average of a distribution of aggregate sizes, but lack any information regarding distribution characteristics and how such aggregates may change over time. Additionally, in current analysis instruments, the suspension of fluid sample which is optically analyzed generally resides in a cuvette or chamber while collimated light passes through a fraction of it. The signal obtained is therefore not an accurate representation of the whole sample volume due to inhomogeneity of the suspension as a result of particles sedimentation and/or improper mixing.

Furthermore, when testing for a target molecule in a blood sample, such as a blood plasma protein, current systems are limited to performing turbimetric assays on a serum sample. As such, an operator is required to first separate serum from a whole blood sample using a centrifuge or filtration device, thus making the process much more labor intensive and not suitable for untrained operators or poor resource settings. Moreover, the measured concentration of blood plasma protein in whole blood specimens needs to be translated to that in serum. In this case, either the hematocrit is estimated or is measured by way of a different device. Furthermore, some analysis instruments require lysing of cells in a fluid sample undergoing analysis. However, lysing of cells may introduce debris and intra-cellular proteins into the fluid sample, which may interfere with the antigen-antibody reactions and further interfere with light measurements, thereby reducing accuracy of turbimetric assays.

Additionally, current analysis instruments are further limited in that they are generally configured to measure a single analyte per cartridge/strip loaded into the instrument, and, in most cases, are limited to running a single assay. For example, it is clinically important to be able to have an overall characterization of the fluid sample, such as a hematocrit, complete blood count (CBC), or the like, in addition to one or more immunoassay measurements, such as a c-reactive protein (CRP) measurement. However, due to the limitations of current analysis instruments, non-immune response assays and immunoassays must be performed on different platforms using different technologies. Furthermore, current analysis instruments are unable to perform multiplexing. In other words, current instruments are unable to measure the concentration of several analytes or target molecules simultaneously.

### SUMMARY

The present invention recognizes the drawbacks of current analysis instruments and provides systems and methods for performing one or more immunoassays on a fluid sample and performing image analysis on the fluid sample as the sample is flowing so as to obtain measurements related to one or more target analytes based on image analysis. Particularly, aspects of the present invention provide system and methods for performing one or more immunoassays using image analysis and flow, as well as an ability to perform assays (such as immunoassays) on a fluidic device (e.g., a disposable cartridge) facilitating use at the point-of-care (POC). The unique combination of image analysis, microfluidics, immunoturbidimetry and innovative fluidic devices (e.g., cartridges) solves the above problems.

Aspects of the invention are accomplished by drawing a sample, for example using a disposable dispenser, and injecting the sample into a first reagent compartment residing on a fluidic device. The sample is mixed with a first reagent and the resulting suspension is flowed into another chamber to react with a subsequent reagent and so forth until the sample is ready for analysis. The suspension of cells (optional) and beads is flowed through a translucent measurement chamber where images of the flowing particles are captured via magnifying optics and a camera. The images of the gradually aggregating particles are analyzed on the fly using image processing algorithms. The cells may be classified using machine learning algorithms and differentiated from the particles. The sizes of the particles are monitored as well as their colors or morphology (for multiplexing purposes) and thus the dynamics of aggregation are recorded. From these measurements the concentration of several analytes can be deduced as well as cell concentration.

In certain embodiments, constant flow allows measuring a large portion of the suspension and thus higher accuracy and repeatability are achieved. The imaging-based analysis allows monitoring the particles aggregation without cells interference by inspecting the space between the cells, thereby disregarding the cells. The complete size distribution at any given time is attained, which provides more information on the reaction. Finally, image analysis enables multiplexing several assays by using colored beads or differently size or shaped beads.

The hematocrit can be assessed from the number of cells counted divided by the volume inspected multiplied by the dilution ratio. Alternately, the hematocrit could be measured via a parallel route in a fluidic device, such as described herein.

In certain embodiments, the systems and methods of the invention include use of a fluidic device (optionally disposable), such as a cartridge, configured to be loaded with a fluid sample and perform one or more assays on the fluid sample. The cartridge is further configured to flow a volume of fluid sample, undergoing, or having undergone, an assay, through a portion thereof to be subsequently analyzed by an analysis instrument. The analysis instrument is configured to capture images of the fluid sample as it is flowing through a portion of the cartridge and subsequently analyze the images so as to obtain measurements of one or more target analytes within the fluid sample.

In one embodiment, at least one particle-based immunoassay may be performed on a fluid sample loaded into the cartridge. The fluid sample may be injected into at least a first reservoir of the cartridge containing a first reagent, upon which mixing occurs. The resulting suspension may then be moved into a second reservoir containing a plurality of particles, each including an antibody that is specific to a target analyte within the fluid sample such the plurality of particles and target analyte will bind to each other, via the antibody, and form one or more aggregates. The suspension of fluid sample and particles (bound to the target analyte) is flowed out of the second reservoir and through a channel of the cartridge. The analysis instrument is configured to capture a plurality of images of the suspension of fluid sample flowing through the channel of the cartridge so as to capture dynamics of formation of the one or more aggregates. The cartridge is able to provide relatively constant flow of the suspension of fluid sample as images are being taken. The constant flow allows for a large portion of the suspension fluid to be measured and ensures that the suspension fluid is relatively uniform during image capture, and thus higher accuracy and repeatability are achieved, in contrast to current analysis instruments, which perform turbimetric assays and analysis on relatively static and non-uniform suspension fluid, resulting in particle sedimentation and/or inhomogeneous suspension.

The analysis instrument is configured to analyze the images, and thereby analyze, on the fly, the dynamics of formation of the one or more first aggregates to determine a concentration of the target analyte in the fluid sample. In particular, image analysis may include obtaining a plurality of different images, wherein dynamics of formation of the one or more aggregates is analyzed in each image, such that the dynamics of formation can be analyzed over a period of time. The dynamics of formation of aggregation may include, but is not limited to, a rate of formation of the one or more aggregates and a size of the one or more aggregates. The analysis instrument is configured to monitor, not only the size and rate of formation of the aggregates, but further monitor one or more characteristics of the particles, such as color, luminescence, size, and/or shape. As such, systems and methods of the invention allow for multiple immunoassays to be simultaneously performed on a fluid sample such that different target analytes may be detected and their associated concentrations may be measured, as different particles may have different characteristics, such as color or morphology (e.g., a first set of particles to bind to a first target analyte have a first color and a second set of particles to bind to a second target analyte have a second color). This multiplexing ability is particularly important in the diagnosing of certain infection and disease states, such as bacterial infection, cancer, or heart failure, as the combination of several biomarkers provides much better sensitivity than each one alone.

The analysis instrument may utilize a specialized algorithm during the image analysis process, in which cells within a fluid sample (i.e., red blood cells, white blood cells, bacterial cells, etc.) may be classified and differentiated from the plurality of particles. Accordingly, the analysis instrument may be configured to differentiate between intact cells and the particles within the suspension of fluid sample. As such, the systems and methods of the invention further allow for additional assays to be performed on the fluid sample (e.g., non-immune response assays) so as to obtain measurements related to specific components within the fluid sample (i.e., cell counting and characterization). For example, a whole blood sample may be loaded into the cartridge, without having to first be separated into a sample of blood serum, and undergo two different assays, such as a complete blood count assay and an immunoassay. Accordingly, the systems and methods allow for multiple assays to be performed on a fluid sample so as to obtain a comprehensive characterization of the fluid sample that is otherwise unavailable with current analysis instruments.

One aspect of the invention provides a method of analyzing a fluid sample. The method includes performing a particle-based immunoassay on a fluid sample that is flowing through a channel and performing image analysis of the flowing fluid sample to analyze dynamics of aggregation of the particles within the flowing fluid sample to determine a concentration of a target analyte in the fluid sample.

In some embodiments, performing image analysis includes obtaining a plurality of different images, wherein dynamics of formation of the one or more first aggregates is analyzed in each image. The step of performing image analysis may include obtaining a vertical scan along a height of the channel. The dynamics of formation of aggregation may include a rate of formation of the one or more aggregates, a size of the one or more aggregates, and a combination thereof.

In some embodiments, the step of performing the particle-based immunoassay on the fluid sample further includes providing a first plurality of particles and a second plurality of particles that comprise an optical characteristic that is different from the first plurality of particles. Each particle of the first plurality of particles comprises a first antibody that is specific to a first target analyte and the first plurality of particles and the first target analyte will bind each other, via the first antibody, to form one or more first aggregates. Each particle of the second plurality of particles comprises a second antibody that is specific to the second target analyte, and the second plurality of particles and the second target will bind each other, via the second antibody, to form one or more second aggregates. The step of performing image analysis of the flowing fluid sample may further include imaging the flowing incubated fluid sample to capture dynamics of formation of the one or more first aggregates and one or more second aggregates and analyzing the dynamics of formation of the one or more first aggregates and the one or more second aggregates to determine a concentration of the first target analyte in the fluid sample and the second target analyte in the fluid sample.

In some embodiments, the fluid sample may include intact cells and the method is conducted in the presence of the intact cells. The image analysis may exclude the intact cells that are present in the imaged fluid sample. The intact cells are excluded by a technique including processing an image of intact cells to produce a background threshold, processing an image of the fluid sample comprising the intact cells and one or more aggregates, and normalizing the image of the fluid sample against the background threshold, thereby excluding intact cells from the image analysis of the fluid sample.

It should be noted, however, that, in some embodiments, the fluid sample may undergo a lysing procedure, which may be useful in measuring intra-cellular proteins, such as HbA1C. Accordingly, the image analysis may exclude cellular debris, while still accounting for the target analyte, namely the intra-cellular protein, in order to determine a concentration of a target analyte in the fluid sample.

In some embodiments, the performing step includes providing a cartridge, introducing the fluid sample comprising a first target analyte into a reservoir of the cartridge, the reservoir comprising a first reagent, and incubating the fluid sample with the first reagent. The performing step further includes flowing the fluid sample to a second reservoir of the cartridge comprising a first plurality of particles, wherein each particle of the first plurality of particles comprises a first antibody that is specific to the first target analyte and the first plurality of particles and the first target analyte will bind each other, via the first antibody, to form one or more first aggregates, and flowing the fluid sample and first plurality of particles through a channel in the cartridge. The method further includes imaging the flowing fluid sample to capture dynamics of formation of the one or more first aggregates, and analyzing the dynamics of formation of the one or more first aggregates to determine a concentration of the first target analyte in the fluid sample.

Another aspect of the invention provides a method of analyzing a fluid sample. The method includes incubating a fluid sample comprising a first target analyte and a first plurality of particles, wherein each particle of the first plurality of particles comprises a first antibody that is specific to the first target analyte and the first plurality of particles and the first target analyte will bind each other, via the first antibody, to form one or more first aggregates, flowing the incubated fluid sample through a channel, imaging the flowing incubated fluid sample to capture dynamics of formation of the one or more first aggregates, and analyzing the dynamics of formation of the one or more first aggregates to determine a concentration of the first target analyte in the fluid sample.

In some embodiments, the imaging step includes obtaining a plurality of different images, wherein dynamics of formation of the one or more first aggregates is analyzed in each image. In some embodiments, the imaging step includes obtaining a vertical scan along a height of the channel.

The dynamics of formation of the one or more aggregates may include a rate of formation of the one or more aggregates, a size of the one or more aggregates, and a combination thereof.

In some embodiments, the fluid sample includes a second target analyte and the incubating step further includes a second plurality of particles, wherein the second plurality of particles comprise an optical characteristic that is different from the first plurality of particles, each particle of the second plurality of particles comprises a second antibody that is specific to the second target analyte, and the second plurality of particles and the second target will bind each other, via the second antibody, to form one or more second aggregates. The method may further include imaging the flowing incubated fluid sample to capture dynamics of formation of the one or more second aggregates and analyzing the dynamics of formation of the one or more second aggregates to determine a concentration of the second target analyte in the fluid sample.

In some embodiments, the fluid sample includes intact cells and the method is conducted in the presence of the intact cells. Accordingly, the analyzing step may exclude the intact cells that are present in the imaged fluid sample. The intact cells may be excluded by a technique that includes processing an image of intact cells to produce a background threshold, processing an image of the fluid sample includes the intact cells and one or more first aggregates, and normalizing the image of the fluid sample against the background threshold, thereby excluding intact cells from the analysis of the fluid sample.

The fluid sample may include whole blood and the target may include, but is not limited to, a c-reactive protein (CRP), HbA1C, PCT, BNP, and a combination thereof.

Another aspect of the invention provides a method for analyzing a fluid sample. The method includes providing a fluidic device includes a first portion configured for performing a complete blood count assay and a second portion for performing an immunoassay, performing the complete blood count assay in the first portion of the fluidic device to obtain a hematocrit, and performing the immunoassay in the second portion of the fluidic device, wherein the obtained hematocrit is used in the analysis of results of the immunoassay. In some embodiments, the immunoassay is performed on a flowing fluid sample.

In some embodiments, the immunoassay is performed using image analysis to analyze dynamics of formation of aggregates in the fluid sample. The immunoassay may be performed on whole blood includes intact cells. The immunoassay may be performed without lysing the intact cells. The image analysis may exclude the intact cells that are present in the imaged fluid sample. The intact cells may be excluded by a technique includes processing an image of intact cells to produce a background threshold, processing an image of the fluid sample includes the intact cells and one or more aggregates, and normalizing the image of the fluid sample against the background threshold, thereby excluding intact cells from the image analysis of the fluid sample.

In some embodiments, the fluidic device is a cartridge that is configured to be operably coupled to an analytical instrument. The cartridge may be pre-loaded with reagents for each of the complete blood count assay and the immunoassay.

The immunoassay may be performed to determine a concentration of a target analyte in the fluid sample, wherein the target analyte includes, but is not limited to, a c-reactive protein (CRP), HbA1C, PCT, BNP, and a combination thereof.

Another aspect of the invention provides a fluid cartridge. The fluid cartridge includes one or more reservoirs includes a reagent for an immunoassay and a first plurality of particles, wherein each particle of the first plurality of particles includes a first antibody that is specific to a first target analyte in a fluid sample, a seal between the one or more reservoirs, and a first channel operably coupled to the one or more reservoirs to receive and flow fluid from the one or more reservoirs. In some embodiments, the one or more reservoirs may further include magnetic particles.

In some embodiments, at least one of the one or more reservoirs includes a deformable cover that can be deformed into one or more pre-threshold and post-threshold configurations, and the seal is configured to burst only when the deformable cover is in one of the plurality of post-threshold configurations.

In some embodiments, the cartridge further includes a first reservoir includes an immunoassay buffer, a second reservoir includes the first plurality of particles that is fluidically coupled to the first reservoir, and at least a third reservoir associated with an inlet that is different from an inlet to the first reservoir and the second reservoir. The third reservoir may include one or more reagents for performing a complete blood count assay. In some embodiments, the fluid cartridge includes a second channel operably coupled to the third reservoir to receive and flow fluid from the third reservoir. The cartridge may be configured such that the first channel and the second channel are coupled to a common junction that is downstream from the first, second, and third reservoirs. The cartridge may be configured such that when fluid flow through the first channel arrives at the common junction, the fluid flow from the first channel displaces and reverses the fluid flow from the second channel. In some embodiments, the cartridge further includes a third channel coupled to the common junction. The cartridge may be configured to be operably coupled to an analytical instrument configured to perform image analysis on fluid sample flowing through the third channel.

### BRIEF DESCRIPTION OF THE DRAWINGS

FIG. 1 is a diagrammatic illustration of a system for analysis of a fluid sample using a cartridge and analysis system according to some embodiments of the present disclosure.

FIG. 2 is a diagrammatic illustration of a cartridge and associated sampler according to some embodiments of the present disclosure.

FIG. 3 is a diagrammatic illustration of the sampler introduced into the cartridge of FIG. 2.

FIG. 4 is a diagrammatic exploded view of a sampler and an associated cartridge according to some embodiments of the present disclosure.

FIG. 5 is a diagrammatic top view of the sampler introduced into the cartridge of FIG. 4 illustrating areas in which a cover film is welded to a rigid base portion of the cartridge.

FIG. 6 is a diagrammatic perspective view of the sampler introduced into the cartridge of FIG. 4.

FIG. 7 is a diagrammatic top view of the sampler introduced into the cartridge of FIG. 4.

FIG. 8 is a block diagram representation of an analysis system according to some embodiments of the present disclosure.

FIG. 9 is a diagrammatic, side view representation of selected internal components of an analysis system, generally embodied as an analysis instrument, according to exemplary embodiments of the present disclosure.

FIG. 10 is a diagrammatic, perspective view representation of the selected internal components of the analysis system of FIG. 9.

FIG. 11 is a diagrammatic representation of an activation module or unit of the analysis system of FIG. 9, according to exemplary disclosed embodiments.

FIG. 12 is a schematic, perspective view of a section of a channel with suspended cells flowing therein as part of a complete blood count (CBC) assay, according to some embodiments of the present disclosure.

FIG. 13 is a schematic, perspective view of a section of a channel of the cartridge with suspended cells and particles flowing therein as part of a particle-based immunoassay, according to some embodiments of the present disclosure.

FIG. 14 is a schematic illustration of a fluid analysis system capturing images of a fluid sample flowing through the channel of the cartridge, according to exemplary embodiments of the present disclosure.

FIGS. 15A, 15B, and 15C are images of aggregation of particles within a fluid sample, undergoing a particle-based immunoassay and absent cells, flowing through a channel of the cartridge, wherein each image is captured at a different respective time period.

FIGS. 16A, 16B, and 16C are images of aggregation of particles within a fluid sample, undergoing a particle-based immunoassay and including intact cells, flowing through a channel of the cartridge, wherein each image is captured at a different respective time period.

FIG. 17 is a graphical representation illustrating dynamics of aggregation of particles over a period of time.

FIGS. 18 and 19 are graphical representations illustrating the accuracy of c-reactive protein measurements performed in accordance with the image-based analysis systems and methods of the present disclosure as compared to existing c-reactive protein measurements obtained via existing analysis platforms.

FIG. 20 is a flow diagram illustrating one embodiment of a method for analyzing a fluid sample.

FIG. 21 is a flow diagram illustrating another embodiment of a method for analyzing a fluid sample.

FIG. 22 is a flow diagram illustrating another embodiment of a method for analyzing a fluid sample.

### DETAILED DESCRIPTION

The present invention provides systems and methods for performing one or more immunoassays on a fluid sample and performing image analysis on the fluid sample as the sample is flowing so as to obtain measurements related to one or more target analytes based on image analysis. Particularly, aspects of the present invention provide system and methods for performing one or more immunoassays using image analysis and flow, as well as an ability to perform assays (such as immunoassays) on a fluidic device (e.g., a disposable cartridge) facilitating use at the point-of-care (POC). The unique combination of image analysis, microfluidics, immunoturbidimetry and innovative fluidic devices (e.g., cartridges) solves the above problems.

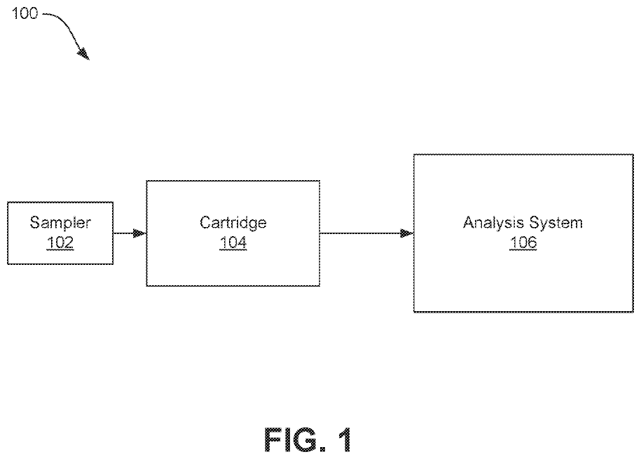

FIG. 1 is a diagrammatic illustration of a system 100 for analysis of a fluid sample. For example, the system 100 may be usable as a Point of Care Testing (POCT) system which enables quick obtaining of laboratory results in a doctor's office. The system 100 generally includes a sampler 102, used for drawing fluid sample therein, and a disposable fluidic device, such as a cartridge 104, configured to interact with the sampler 102 to thereby receive the fluid sample therefrom. The cartridge 104 prepares the fluid sample for analysis via the analysis system 106. In particular, the cartridge 104 is configured to perform one or more assays on the fluid sample. The cartridge is further configured to flow a volume of fluid sample, undergoing, or having undergone, an assay, through a portion thereof to be subsequently analyzed by the analysis system 106. The analysis system 106 is generally configured to capture images of the fluid sample as it is flowing through a portion of the cartridge 104 and subsequently analyze the images so as to obtain measurements of one or more target analytes/molecules and/or cells within the fluid sample.

The fluid sample may generally contain cells and/or target analytes for analysis. The cells may be any type of prokaryotic cells, including, but not limited to, bacteria, eukaryotic cells, such as red blood cells, white blood cells (Leukocytes), epithelial cells, circulating tumor cells, cellular fragments, for example platelets, or others. The target analytes may include, but are not limited to, c-reactive protein (CRP), HbA1C, procalcitonin (PCT), brain natriuretic peptide (BNP), or any other target analyte or molecule that may be indicative of a condition or disease.

Accordingly, at least one particle-based immunoassay may be performed on a fluid sample loaded into the cartridge 104. The fluid sample may be injected into at least a first reservoir of the cartridge 104 containing a first reagent, upon which mixing occurs. The resulting suspension may then be moved into a second reservoir of the cartridge 104 containing a plurality of particles, each including an antibody that is specific to a target analyte within the fluid sample such the plurality of particles and target analyte will bind to each other, via the antibody, and form one or more aggregates. The suspension of fluid sample and particles (bound to the target analyte) is flowed out of the second reservoir and through a channel of the cartridge 104, such as a translucent measurement channel. The analysis system 106 is configured to capture a plurality of images of the suspension of fluid sample flowing through the channel of the cartridge 104 so as to capture dynamics of formation of the one or more aggregates. The cartridge 104 is able to provide relatively constant flow of the suspension of fluid sample as images are being taken, either by way of a pump (not shown) or other means. The constant flow allows for a large portion of the suspension fluid to be measured and ensures that the suspension fluid is relatively uniform during image capture, and thus higher accuracy and repeatability are achieved.

The analysis system 106 is configured to analyze the images, and thereby analyze, on the fly, the dynamics of formation of the one or more aggregates to determine a concentration of the target analyte in the fluid sample. In particular, image analysis may include obtaining a plurality of different images, wherein dynamics of formation of the one or more aggregates is analyzed in each image, such that the dynamics of formation can be analyzed over a period of time. The dynamics of formation of aggregation may include, but is not limited to, a rate of formation of the one or more aggregates and a size of the one or more aggregates. The analysis system 106 is configured to monitor, not only the size and rate of formation of the aggregates, but further monitor one or more characteristics of the particles, such as color, size, and/or shape. As such, systems and methods of the invention allow for multiple immunoassays to be simultaneously performed on a fluid sample such that different target analytes may be detected and their associated concentrations may be measured, as different particles may have different characteristics, such as color or morphology (e.g., a first set of particles to bind to a first target analyte have a first color and a second set of particles to bind to a second target analyte have a second color). This multiplexing ability is particularly important in the diagnosing of certain infection and disease states, such as bacterial infection, cancer, or heart failure, as the combination of several biomarkers provides much better sensitivity than each one alone.

The analysis system may utilize a specialized algorithm during the image analysis process, in which cells within a fluid sample (i.e., red blood cells, white blood cells, bacterial cells, etc.) may be classified and differentiated from the plurality of particles. Accordingly, the analysis system may be configured to differentiate between intact cells and the particles within the suspension of fluid sample.

As such, the system 100 allows for at least one additional assay to be performed on the fluid sample (e.g., non-immune response assay) so as to obtain measurements related to specific components within the fluid sample (i.e., cell counting and characterization). For example, a whole blood sample may be loaded into the cartridge, without having to first be separated into a sample of blood serum, and undergo two different assays, such as a complete blood count assay and an immunoassay.

The following description refers to the fluid sample as being a whole blood sample, and the cartridge is used in preparing and performing multiple assays on the whole blood sample. For example, in the following description, a whole blood sample may be loaded into the cartridge, without having to first be separated into a sample of blood serum, and undergo two different assays, such as a complete blood count (CBC) assay to obtain CBC measurements, including a hematocrit (Hct) measurement, and a particle-based immunoassay to obtain a concentration measurement of a target analyte from the immunoassay, such as a c-reactive protein (CRP) measurement.

It should be noted, however, that the present disclosure is not limited to CBC and CRP measurements. The systems and methods of the present disclosure may be used for multiple applications where analysis of cells and/or target analytes is desired, such as detecting cancer, heart failure, diabetes detection and monitoring, thrombosis diagnosis (D-dimer), HIV detection and monitoring (such as using CD4/CD8 ratio), detection of f-hemoglobin, Feritin, Malaria antigen or other blood parasites, Paroxysmal Nocturnal Hemoglobinuria (PNH), diagnosis of Celiac disease using intestinal Endomysial Autoantibodies (EmA). Alzheimer's disease, or any other application for which target analyte/molecule and/or cell-based diagnosis may be relevant.

It should be noted that various embodiments of a sampler, cartridge, and a compatible diagnostic instrument for analyzing the fluid sample, and methods of use are described in at least U.S. Pat. Nos. 9,222,935; 9,404,917; and 9,592,504; and 9,683,984, the contents of each of which are hereby incorporated by reference in their entireties.

### Disposable Cartridge

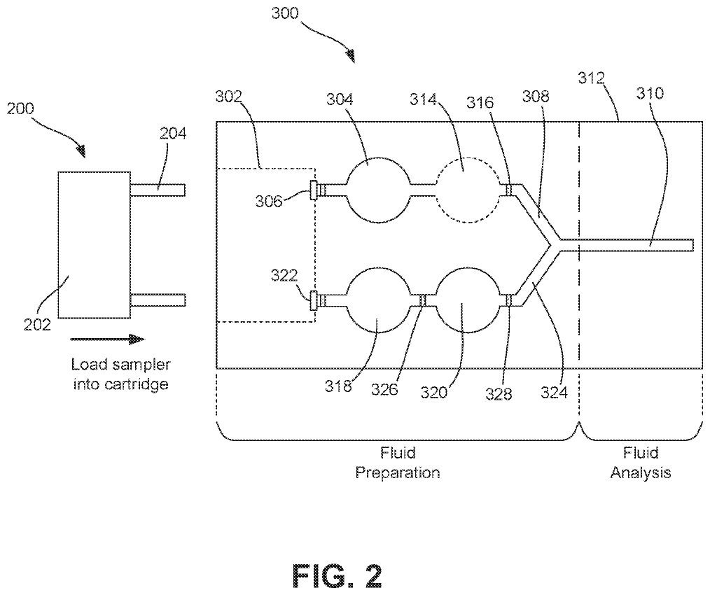

FIG. 2 is a diagrammatic illustration of a sampler 200 and an associated cartridge 300 according to some embodiments of the present disclosure. The sampler 200 may function to introduce a fluid sample into the cartridge 300. As shown, the sampler 200 includes two capillaries 204 attached to a handle member 202. However, it should be noted that a sampler may include any number of capillaries, including a single capillary, or more than two capillaries. Each capillary is able to draw a fluid sample within by way of capillary action.

The fluid sample may generally contain cells and/or target analytes for analysis. It should be noted that the fluid sample may include biological sample of any kind, including a human bodily fluid, and may be collected in any clinically acceptable manner. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CSF. A sample also may be media containing cells or biological material. A sample may also be a blood clot, for example, a blood clot that has been obtained from whole blood after the serum has been removed. In certain embodiments, the sample is blood collected from a subject.

Inside the capillary 204, a seal/plug may be formed, and the seal or plug may include any type of material or configuration that allows at least some air to flow, but blocks liquid flow. For example, in some embodiments a venting plug (not shown) may be affixed at a pre-determined distance from the capillary outlet. The capillary 204 may include any type of capillary with a venting plug affixed inside and suitable for a particular application. For example, capillaries manufactured by DRUMMOND Aqua-Cap™ Microdispenser may be used in the presently disclosed embodiments.

Fluid sampling may be performed by immersing the outlet of the capillary 204 in the fluid. The fluid sample may be driven into the capillary by capillary force. The venting plug affixed inside the capillary 204 may facilitate the process, as it allows the air displaced by the fluid sample to flow out. The fluid fills the capillary until reaching the venting plug. It should be appreciated that the plug may be porous and hydrophobic or hygroscopic so that, once in contact with the fluid, the plug becomes nearly impassable to fluid flow. Therefore, there may be no fluid sample absorbance in the plug, or in other words, no loss of fluid volume occurs to the plug nor will there be reagent leakage through the plug in the subsequent stage. Thus, the final volume of a sampled fluid may be determined based on a distance of the venting plug from the capillary outlet and by the capillary's inner diameter.

Upon drawing a fluid sample into the capillaries 204, the sampler 200 may then be introduced into the cartridge 300 (e.g., from one side). In particular, the cartridge 300 may include a receiving portion 302 shaped and/or sized to receive at least the capillaries 204 within. As shown, however, the receiving portion 302 may receive a majority of the sampler 200 within, including the handle member 202 and capillaries 204. The receiving portion 302 may further include a means of retaining the sampler 200 within, such as, for example, a snap-fit connection (cooperative locking and tab members), press-fit connection, and the like. For example, when sampler 200 is introduced into the receiving portion 302 of the cartridge 300, a deflection tab (not shown) on the sampler 200 may cause deflection of a locking tab (not shown) within the receiving portion 302, such that continued movement of sampler 200 into the receiving portion 302 may release the locking tab from its deflected position allowing the locking tab to snap into place behind the advancing deflection tab. The deflection tab and the locking tab may be shaped such that the deflection tab can pass the locking tab only in one direction. Thus, once sampler 200 is introduced fully into the receiving portion 302, interference between the locking tab and deflection tab may prevent sampler from being removed from the cartridge 300.

The cartridge 300 may generally include at least two sections, including a preparation section and an analysis section. The fluid sample may first be introduced into the preparation section (from the sampler 200), in which one or more processes may be performed relative to the fluid sample to prepare the fluid sample for analysis. The analysis section may then receive the prepared fluid sample (from the preparation section) and may enable analysis of one or more aspects of the fluid sample. In some embodiments, the preparation and analysis sections may be separately formed and coupled together by one or more flow paths. In such an embodiment, the analysis section may be referred to an analysis chip 312. In some embodiments, the preparation and analysis sections may be manufactured together and coupled during, or immediately after manufacturing, or they may be manufactured separately and become coupled prior to marketing the cartridge to its end user or even just prior to usage thereof, possibly even by a person performing the test or automatically inside system 100. In some embodiments, for example, the preparation and analysis sections may be integrally formed relative to a common substrate.

As shown, the cartridge 300 may include multiple reservoirs for receiving a fluid sample from the sampler 200 and further prepare the fluid sample for analysis (i.e., performing one or more assays on the fluid sample within the reservoir). The preparation, performed inside the one or more reservoirs may include any procedure that may provide a change of a physical or a chemical state (or a change of at least one property or characteristic) of the fluid sample or of cells and/or target analytes/molecules contained within the fluid sample. Examples of possible affecting procedures may include heating, mixing, diluting, staining, permeabilization, lysis, etc. Some of the procedures will be described below with reference to the following figures.

In certain embodiments of the disclosure, the reservoirs may be pre-loaded with a substance. The pre-loaded substance may be a liquid substance, a solid substance, or a combination thereof. The substance may consist of a single reagent or of several different reagents. An example of a liquid substance consisting of several reagents is PBS (Phosphate Buffered Saline), while examples of solid substances are lyophilized antibodies, different kinds of powdered stains dissolvable, e.g., in water or in ethanol, coated beads, etc. A substance may be lying free on the bottom of the reservoir or may be attached to an inner surface of the reservoir. Alternatively, a substance may be attached to structures or components, such as sponge or microfibers, filling the space of the reservoir. Such structures or components may enlarge an amount of surface area exposed to the fluid sample.

Furthermore, some possible procedures, such as heating, do not require having a pre-loaded substance in the reservoir. Therefore, in certain embodiments, the reservoir is not pre-loaded with a substance, while it is possible that the reservoir holds instead (or in addition to a pre-loaded substance) a mechanism, such as a heating mechanism or part thereof. In addition, understanding that pre-loading the substance may be performed during manufacturing of the cartridge or at any time prior to the introduction of the fluid sample, in alternative embodiments, the substance may be introduced into the reservoir together with or after introducing the fluid sample. In other embodiments, wherein the substance is composed of a combination of constituents or wherein the substance is the outcome of a chemical reaction between more than one constituents, it is possible that at least one constituent is pre-loaded while at least one other constituent is introduced with or after introduction of the fluid sample.

In case a reservoir is loaded with a substance, whether pre-loaded or loaded with/after introduction of the fluid sample, the procedure affecting the fluid sample may include mixing of the fluid sample with the substance. In some cases, the fluid sample and the substance may be mixed thoroughly as a lack of homogeneity may impact subsequent analysis. According to certain embodiments of the disclosure, in order to enable mixing, at least part (a portion) of the surface of the reservoir, may include a deformable or pressable portion (such as a cover or cap over the reservoir. The deformable portion may be made of an elastic polymer, for example, polyurethane or silicone, or of a different elastic material. Due to deformation (such as constriction) of the reservoir, affected by pressing and/or releasing the deformable portion, fluid contained within the reservoir may form a jet flow inside the reservoir, which is a form of flow that may enhance mixing. As such, it may be possible to achieve mixing by alternatively pressing and releasing the deformable portion of the reservoir. When the deformable portion is pressed, the fluid may flow away from the pressed area, and when it is released, the fluid may flow back, such that the fluid flows back and forth.

It should be noted that mixing may occur due to other forces. For example, in some embodiments, the reservoir may include magnetic particles, such that, upon application of a magnetic field (i.e., from an external source), the magnetic particles may move within the reservoir to cause mixing of the fluid sample.

Apart from or in addition to mixing, procedures affecting the fluid sample performed in the reservoir may include reactions that may occur between the substance and the fluid sample. The reaction may include a chemical reaction, for example oxidation/reduction, or a biochemical reaction such as binding antibodies to antigens. The procedure may lead to changes in physical and/or chemical states of the fluid sample or of cells contained within the fluid sample. For example, it may affect changes in viscoelastic properties or in pH of the fluid sample. A concentration of cells contained in a fluid sample may decrease due to dilution. A cellular membrane may become permeable enabling binding of coloring agents or antibodies contained within the substance to cellular components, such as cytoplasmic granules. An oxidation or reduction of different cellular components may happen, such as oxidation of hemoglobin contained in the red blood cells into methemoglobin, etc.

Upon completion of (or at least initiation of) the preparation procedure, the resulting fluid may be released from the reservoir to undergo analysis. The releasing may be affected by positive pressure or “pushing” the fluid out of the reservoir. For example, fluid may be pushed out of the reservoir by applying a force upon the deformable cover of the reservoir into a post-threshold configuration to thereby break a seal and allow fluid to flow out of the reservoir and into an associated channel downstream from the reservoir.

Additionally or alternatively, the fluid may be affected by negative pressure, for example if fluid is driven out of the reservoir by physical forces the “pull” it out, such as gravitational force or due to application of external forces such as a vacuum. In certain embodiments of the disclosure, the flow of the output fluid from the reservoir and into an associated downstream channel may be caused by a suction force generated by a vacuum pump, for example.

The fluid may then travel into a channel or chamber of the analysis section of the cartridge 300, in which the fluid is analyzed, via an analysis system, as the fluid is flowing through a channel or chamber of the analysis section of the cartridge 300.

In the illustrated embodiment, the cartridge 300 includes at least a first reservoir 304 including an inlet 306 to which a first capillary 204 of the sampler 200 is to be coupled, and a pair of reservoirs connected in series (i.e., second reservoir 318 and third reservoir 320) including an inlet 322 to which a second capillary 204 of the sampler 200 is to be coupled. In this embodiment, the cartridge 300 is configured to prepare separate volumes of fluid sample (received from the two capillaries 204 of the sampler 200) for analysis. In particular, a non-immune response assay may be performed the fluid sample provided in the first reservoir 304, while a particle-based immunoassay may be performed on the fluid sample provided in the pair of reservoirs (second and third reservoirs 318, 320). For example, the first reservoir 304 may include one or more reagents for performing a complete blood count (CBC) assay, while the second and third reservoirs 318, 320 may include an immunoassay buffer and a plurality of particles, respectively, wherein each particle comprises an antibody that is specific to a target analyte in the fluid sample. In particular, a whole blood sample may be loaded into the respective reservoirs in the cartridge 300, without having to first be separated into a sample of blood serum, and undergo two different assays, such as a complete blood count (CBC) assay to obtain CBC measurements, including a hematocrit (Het) measurement, and a particle-based immunoassay to obtain a concentration measurement of a target analyte from the immunoassay, such as a c-reactive protein (CRP) measurement.

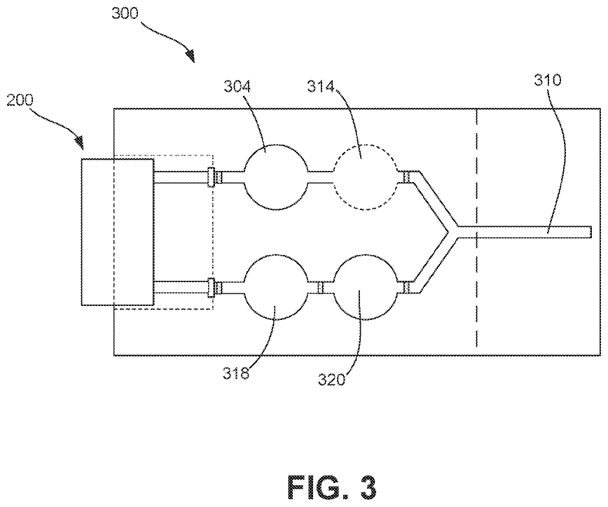

For example, once a fluid sample has been sampled (by the sampler 200), the fluid sample is introduced into the cartridge 300 by inserting the sampler 200 into the receiving portion 302 of the cartridge 300 (as illustrated in FIG. 3). The receiving portion 302 may be shaped and/or sized to align each capillary 204 with the respective inlet 306 and 322 of the reservoirs. At this stage, only a limited leakage of a fluid sample from the capillary into a reservoir may occur, as the fluid may be held inside the capillary by capillary forces. A plunger (not shown) may be used to push the fluid sample out of the capillary and into the respective reservoirs. The plunging member may be configured for insertion into the capillary 204 through a capillary inlet located in the handle member 202. The plunger may push the venting plug until it reaches the capillary outlet, optionally resulting in the delivery of the entire fluid sample into the associated reservoir. As described in at least U.S. Pat. Nos. 9,222,935; 9,592,504; and 9,625,357, a diagnostic instrument, into which a cartridge (including the sampler device coupled thereto) has been loaded, may include a plunger or other mechanism for contacting the plug of a capillary, wherein the plunger is used to push a volume of fluid sample out of the capillary to undergo analysis.

The first reservoir 304 may be enclosed between two seals, wherein the preceding seal (between the reservoir 304 and the inlet 306) prevents fluid from flowing out of the reservoir 304 into the inlet 306 and the succeeding seal 316 prevents fluid from flowing out of the reservoir 304 and into a downstream channel 308. Prior to introduction of the fluid sample into reservoir 304, the seals may prevent release of substances from the reservoir 304. These seals may also prevent release of the substance and/or the fluid sample during preparation of the fluid sample within the reservoir 304, and thus may prevent unintentional release of resulting fluid for analysis. Regarding seal 316, breaking or breaching of seal 316 may allow fluid to flow out of the reservoir 304 towards the downstream channel 308 for analysis within a channel or chamber 310 of the analysis section of the cartridge 300. The seals may constitute breakable or “frangible” seals. For example, it is possible to form the seal (e.g., of adhesive) configured to be to be broken by application of pressure exceeding a certain threshold. Accordingly, applying pressure on the deformable cover of reservoir 304 may result in a pressure at the position of the seal 316 that exceeds the breaking threshold of the seal, which causes the seal to be breached. The fluid may then be released into the downstream channel 308 and into the analyzing section.

However, it should be noted that mixing of the fluid sample with a reagent or buffer within the first reservoir 304 by intermittently pressing the deformable cover of the reservoir 304 may not result in post-threshold pressure at the position of the seal 316. Thus, during mixing, the seal 316 may remain intact. In some embodiments, an ancillary reservoir 3808 may be provided and may be fluidically coupled to the first reservoir 304, such that pressing deformable covers of reservoirs 304 and 314, in a pre-threshold configuration and an alternating pattern, may result in further mixing of the fluid sample, as portions of the fluid sample may move between reservoirs 304 and 314.

The initial seal (provided at the inlet 306 prior to the reservoir 304) may have two different roles. In a first role, the seal may prevent the release of the substance from the reservoir prior to the introduction of the fluid sample. However, when introducing the fluid sample, the preceding seal must be broken, in order to allow such introduction. The introduction of the capillary 204 may result in breaking of the initial seal. The initial seal may be resealable, such that a seal is formed around the capillary, thereby allowing mixing using pressure provided to the deformable portion of the reservoir, as the reservoir can be sealed from both sides after the sampler 200 is coupled to the cartridge 300.

The second reservoir 318 may also be enclosed between two seals, wherein the preceding seal (between the reservoir 318 and the inlet 322) prevents fluid from flowing out of the reservoir 318 into the inlet 322 and the succeeding seal 326 prevents fluid from flowing out of the second reservoir 318 and into the third reservoir 320. For example, the second reservoir 318 and third reservoir 320 may each include substances that must remain separate until a desired reaction is to occur. In this instance, the second reservoir 318 may include an immunoassay buffer and the third reservoir 320 may include a plurality of particles (i.e., micro- or nano-beads), wherein each particle includes an antibody that is specific to a target analyte within the fluid sample such the plurality of particles and target analyte will bind to each other, via the antibody, and form one or more aggregates. The fluid sample may first need to incubate within the immunoassay buffer for a period of time before being mixed with the plurality of particles. Accordingly, seal 326 prevents fluid from the second reservoir 318 from flowing into the third reservoir 320 until the incubation period is complete. As such, a fluid sample may initially be provided into the second reservoir 318 (the initial seal is broken upon coupling of the capillary 204 with the inlet 322), at which point the fluid sample is mixed with the immunoassay buffer and allowed to incubate, whereby seal 326 prevents flow of the mixed fluid into the third reservoir 320.

Mixing of the fluid sample with the immunoassay buffer may be achieved by applying pressure on the deformable cover of reservoir 318 in pre-threshold configuration (i.e., below the breaking threshold of the seal 326). Alternatively, magnetic particles may be provided in reservoir 318, such that mixing occurs as a result of applying a magnetic field upon the magnetic particles. The mixed fluid in reservoir 318 may then be released and allowed to flow into reservoir 320 upon applying pressure on the deformable cover of reservoir 318 in a post-threshold configuration, which results in a pressure at the position of the seal 326 that exceeds the breaking threshold of the seal, which causes the seal to be breached. Accordingly, the fluid may further mix with the plurality of particles in reservoir 320. Again, applying pressure on the deformable cover of reservoir 320 in a post-threshold configuration results in a pressure at the position of the seal 328 that exceeds the breaking threshold of the seal, thereby allowing the suspension of fluid sample and particles to flow into a downstream channel 324 for subsequent analysis in the analysis section of the cartridge 300 (i.e., into channel or chamber 310 of the analysis section).

As shown, the cartridge 3M) may be configured such that the downstream channels (first channel 308 and second channel 324) are coupled to a common junction that is downstream from the first, second, and third reservoirs 304, 318, and 320. Accordingly, the cartridge 300 may be configured such that when fluid flow through the first channel 308 arrives at the common junction, the fluid flow from the first channel 308 displaces the fluid flow from the second channel 324, and vice versa. As such, only one fluid sample is allowed to flow through the channel 310 of the analysis section of the cartridge 300 at any given time, thereby preventing mixing of the fluid samples.

As will be described in greater detail herein, the analysis system, into which the cartridge 300 (having the sampler coupled thereto) is loaded, may include various components and mechanisms for moving fluid sample into and through the cartridge 300, thereby controlling preparation of the fluid samples in respective reservoirs and further controlling flow of prepared fluid samples for subsequent analysis.

It should be noted that a cartridge consistent with the present disclosure may include any number of reservoirs connected in series so as to carry out an immunoassay, or any assay involving multiple reagents or specific stages of preparation in which a fluid sample requires isolation. For example, as illustrated in FIGS. 2 and 3, the cartridge 300 includes reservoirs 318 and 320 connected in series, wherein reservoir 318 includes an immunoassay buffer and reservoir 320 includes a plurality of particles, such that, a fluid sample is first introduced into reservoir 318 and allowed to mix with the immunoassay buffer and incubate for a period of time prior to flowing into reservoir 320 to then be mixed with the plurality of particles.

In some embodiments, the immunoassay may involve lysing the fluid sample, which may be useful when the target analyte is an intra-cellular protein, such as HbA1C. Accordingly, in some embodiments, the cartridge 300 may include at least three reservoirs connected in series, wherein a first reservoir includes a lysing reagent, a second reservoir includes the immunoassay buffer, and the third reservoir includes the plurality of particles. Accordingly, a fluid sample is first introduced into a first reservoir and allowed to mix with a lysing reagent until lysing of cells occurs, then the fluid flows into the second reservoir and allowed to mix with the immunoassay buffer and incubate for a period of time prior to flowing into a third reservoir to then be mixed with the plurality of particles. In yet another embodiment, only two reservoirs may be required, such that reservoir 318 may include both a lysing reagent and an immunoassay buffer and reservoir 320 includes a plurality of particles.

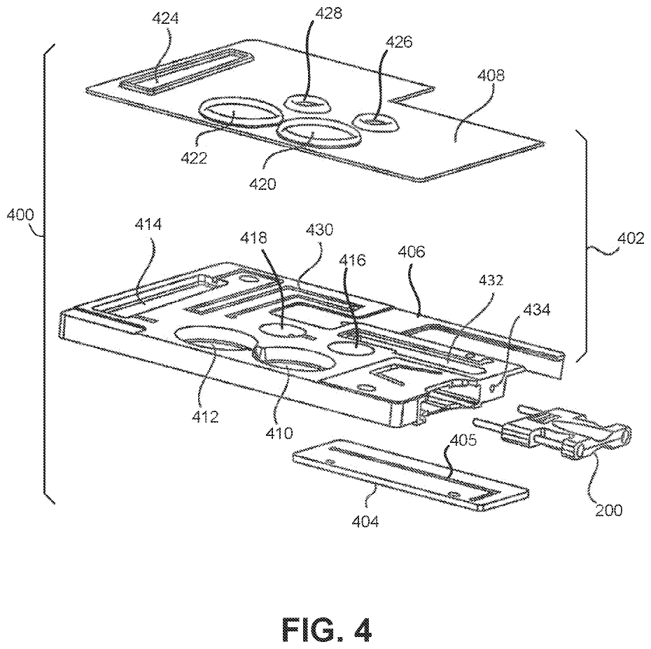

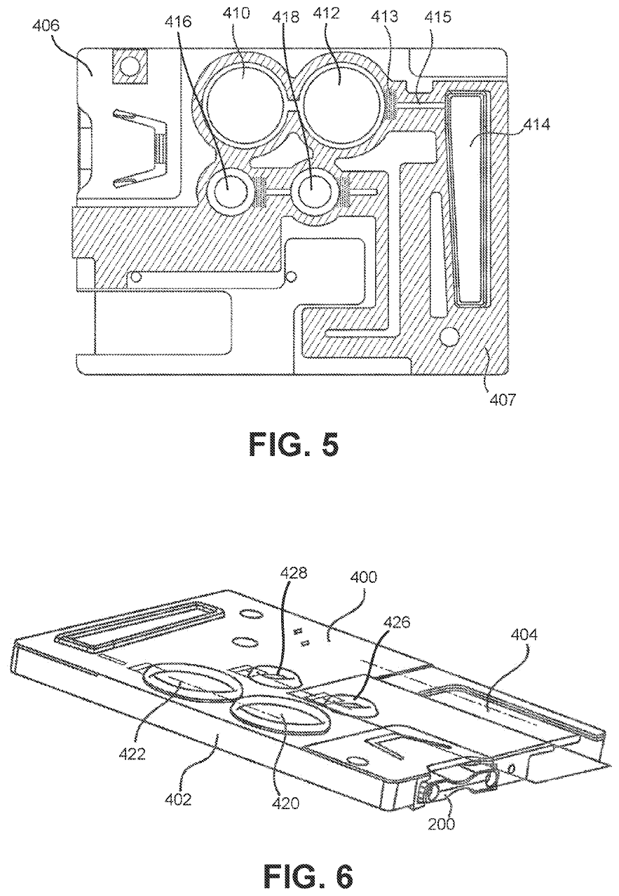

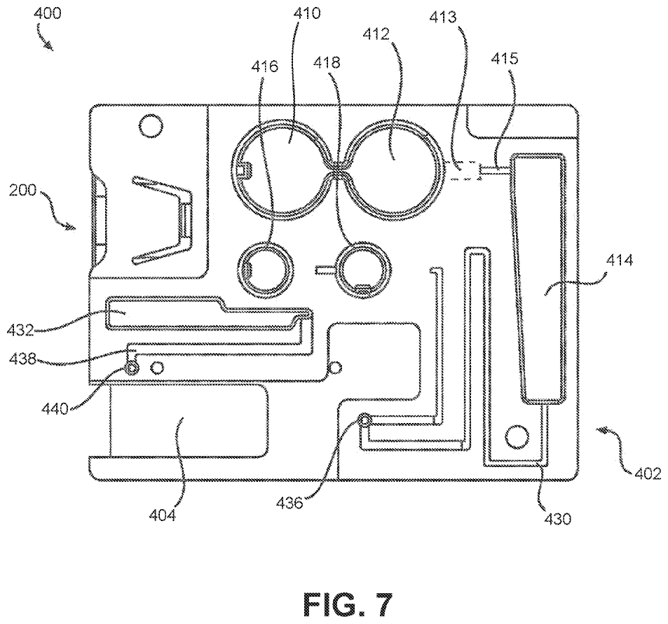

FIG. 4 is a diagrammatic exploded view of a sampler 200 and another embodiment of associated cartridge 400. FIG. 5 is a diagrammnatic top view of the sampler 200 introduced into the cartridge 400, illustrating areas in which a cover film is welded to a rigid base portion of the cartridge 400. FIGS. 6 and 7 are diagrammatic perspective and top views, respectively, of the sampler 200 introduced into the cartridge 400. Cartridge 400 may include a preparation unit 402 and a fluid analysis chip 404 attached to the preparation unit.

Preparation unit 402 may include any suitable structures for receiving a fluid to be analyzed, preparing the received fluid for analysis, and providing the prepared fluid to the fluid analysis chip 404. For example, in some embodiments, preparation unit 402 may have a two-part construction, including, for example, a rigid base portion 406 and a flexible film 408. Rigid base portion 406 and flexible film 408 may be similar to rigid frame 406 and film 408, respectively.

The rigid base portion 406 may comprise any rigid or semi-rigid material. For example, in some embodiments, the rigid base portion 406 may be fabricated from any of PMMA, COP (cyclic olefin copolymer), polyethylene, polycarbonate, polypropylene, polythene, etc., or combinations thereof. The rigid base portion 406 may also be fabricated to include one or more structures associated with any of the preparation units described above. For example, in some embodiments, rigid base portion 406 may be made by injection molding and may include various flow paths, channels, inlets, outlets, and/or reservoir elements (e.g., depressions formed in a surface of the rigid frame that provide reservoirs when covered with a cap or cover layer). Rigid base portion 406 may be provided as a substantially monolithic substrate. In other embodiments, rigid base portion 406 may include more than one component. In some embodiments, rigid base portion 406 may include one or more depressions, such as depressions 410, 412, 414, 416, and 418 formed in a top surface of rigid base portion 406. The depressions may correspond to reservoirs intended to receive a fluid sample and prepare the fluid sample for analysis, as previously described herein. For example, at least depressions 410 and 412 may be similar to, and function similarly as, reservoirs 304 and 314, described with respect to cartridge 300 of FIGS. 2 and 3. Depressions 416 and 418 may be similar to, and function similarly as, reservoirs 318 and 320, described with respect to cartridge 300 of FIGS. 2 and 3.

Preparation unit 402 may be formed by joining flexible film 408 with rigid base portion 406. Film 408 may be formed of from any suitable material. In some embodiments, film 408 may be formed from PVC. PET, polypropylene, polyethylene, polyurethane and laminates containing aluminum and PE, or combinations thereof.

In some embodiments film 408 may be flexible and when attached to rigid base portion 406 may extend over a top surface of rigid base portion 406. Film 408 may include a flat sheet of material. In other embodiments, however, film 408 may include preformed shapes or structures that form either raised or sunken areas in film 408. These raised or sunken areas may be formed in certain areas of film 408 such that when film 408 is joined to rigid base portion 406, the raised or sunken areas overlap with or otherwise correspond to corresponding structures formed in rigid base portion 406. For example, in some embodiments, a raised portion of film 408 (e.g., a cap) may be formed in a location that overlaps with any of depressions 410, 412, 414, 416, or 418. Such overlapping caps and depressions may form fluid reservoirs when film 408 is joined together with rigid base portion 406. Likewise, in some embodiments, sunken portions of film 408 may be formed in locations that overlap with any of depressions 410, 412, 414, 416, or 418. As shown, raised caps 420 and 422 overlap with depressions 410 and 412, respectively. Similarly, raised caps 426 and 428 overlap with depressions 416 and 418, respectively. Also shown is a sunken portion 424 of film 408, which overlaps depression 414. In some embodiments, flexible film 408 covering the rigid base 406 may be pre-formed to a geometry having redundant area to enable stretching, which may facilitate a selective increase and/or decrease of a volume of a reservoir.

Notably, a reservoir may be formed by a single depression in rigid base portion 406 when covered by film 408. For example, reservoir 414, as shown in FIG. 5, may be formed by sunken portion 424 overlapping depression 414. In other embodiments, however, reservoirs may be formed to include more than one depression. For example, depression 410 is connected to depression 412 via a groove formed in the top surface of rigid base portion 406. This groove establishes fluid communication between depression 410 and depression 412, such that when film 408 is joined to rigid base portion 406, a single fluid reservoir is formed by depressions 410 and 412, as covered by caps 420 and 422.

The rigid base portion 406 may include one or more structures for receiving a structure associated with sampler 200. For example, in some embodiments, rigid base portion 406 may include reservoir inlet (not shown). Reservoir inlet may be configured with a size and shape suitable to receive, align, and stabilize a capillary tube associated with sampler 200.

As noted above, preparation unit 402 may be formed by joining film 408 to rigid base 406. Such joining may be accomplished, for example, by any known joining or welding techniques. FIG. 5 provides a diagrammatic top view illustration of one embodiment of a disposable cartridge 400 formed by patterned thermo welding of film 408 to a rigid base portion 406. Areas that have been welded are shown either with a dotted pattern or a cross-hatched pattern. In the embodiment of FIG. 5, the areas of dotted patterning represent temporary, frangible seals, and the areas shown in cross-hatching represent permanent seals.

In some embodiments, one or more of the rigid base 406 and the film 408 may be formed of materials that may bond together when exposed to heat. During construction of the two-part structure of preparation unit 402 (FIG. 4), varying levels of heat may be applied to achieve desired results. For example, where high temperatures (e.g., 140° C.-180° C.) are applied, film 408 may be caused to permanently weld to the material of rigid base 406 (cross-hatched pattern of FIG. 5). In other areas, where little or no heat is applied, film 408 may remain unbonded to the underlying rigid frame. And, in areas where heat is provided at a level below a welding threshold for the materials (e.g., 100° C.-130° C.), the material of film 408 may bond together with the material of rigid base 406, but the bond may be non-permanent (dotted pattern of FIG. 5). That is, in these areas, the bonded materials may be later pulled apart from one another.

In some embodiments, the selective bonding described above may be achieved, for example, using a film 408 having a multi-layer structure. A first sub-film of the multi-layer structure (e.g., the lowest layer that first contacts rigid base 406) may include a material that forms a relatively weak bond with the material of rigid base 406. Thus, subsequent force on an area where the first sub-film has been bonded to rigid base 406 may result in separation (e.g., peeling) of the sub-film and, therefore, the entire film 408 away from rigid base 406.

In some embodiments, a multi-layer structure of film 408 may include a second sub-film disposed above the first sub-film. The second sub-film may form a more permanent bond with the material of rigid base 406 through the application of a higher temperature. For example, in some embodiments, the higher temperature may cause the first sub-film to melt and flow away from the bonding area, which may enable the second sub-film to bond directly to the rigid frame material (either permanently or semi-permanently).

This type of bonding may facilitate construction of components associated with preparation unit 402. For example, in areas such as region 407, a high temperature may be applied to permanently weld the material of film 408 to rigid base 406. In areas associated with reservoirs 410, 412, 414, 416, and 418 and fluid conduit 415, heat application may be avoided such that film 408 remains free of rigid base 408 in these regions. In regions associated with seals (e.g., frangible seal 413), a sub-welding heating level may be used such that film 408 is tacked or temporarily bonded to rigid base 406. These seals may be referred to as “peel seals.” as pressure placed on the seal, for example by a fluid within reservoir 410 pressing on seal, may cause film 408 to peel away from rigid base 406. Under such circumstances, fluid may be allowed to flow through the seal. While these peel seals may be frangible, fluid flow through a broken seal may be halted by, for example, applying pressure to film 408 in the regions of the seals in order to close the fluid pathway at the seals. The peel layers of film 408 may be designed to yield or tear at a specific stress level influenced by polymer composition of film 408 and geometry of the frangible seals.

In addition to layers used in creating frangible seals and/or bonds with rigid base 406, film 408 may also include other layers. For example, film 408 may include one or more layers that serve as barriers for gas and/or moisture permeation. Examples for water vapor barriers include films containing aluminum, aluminum-oxide, or PCTFE. Many of these materials, while being flexible, may exhibit low stretch. Thus, the use of pre-formed raised or sunken structures in film 408 may facilitate fluid movement without reliance upon a need for stretching film 408.

FIG. 6 provides a diagrammatic illustration of a sampler 200 introduced into a cartridge 400, including a preparation unit 402 and a fluid analysis chip 404, according to presently disclosed embodiments. Visible are the raised portions 420 and 422 of film 408 that are used to form reservoirs 410 and 412, as well as raised portions 426 and 428 of film 408 that are used to form reservoirs 416 and 418. Also visible is the sunken portion 424 of film 408 used to form buffer chamber 414. In the embodiment shown in FIG. 6, fluid analysis chip 404 is attached (e.g., bonded) to an underside of preparation unit 402.

Turning to FIG. 7, preparation unit 402 may include a first flow path including at least one fluid conduit 415. This fluid conduit 415 may be formed, for example, by the flexible film 408 extending over one or more grooves 430 (FIG. 4) formed in the top surface of the rigid base portion 406. In some embodiments, this first fluid flow path may be configured to carry a fluid sample including at least the fluid to be analyzed from a reservoir on the preparation unit to a preparation unit fluid outlet 436 enabling the fluid sample to exit preparation unit 402 and enter, for example, fluid analysis chip 404. It should be noted that the fluid sample may include only the fluid to be analyzed as introduced into preparation unit 402 from capillary. In some embodiments, however, the fluid sample carried by the first fluid flow path may include a suspension including the fluid to be analyzed (introduced from capillary) mixed together with one or more fluids included in a reservoir associated with preparation unit 402. For example, at least one of reservoirs 410, 412, and 414 include one or more reagents for performing a complete blood count (CBC) assay on a fluid sample, while reservoirs 416 and 418 include an immunoassay buffer and a plurality of particles, respectively, wherein each particle comprises an antibody that is specific to a target analyte in the fluid sample. As such, cartridge 400 may be used to perform two separate assays on a fluid sample.

The first flow path may include structures other than fluid conduit 415. For example, the first fluid flow path may include a buffer chamber 414 formed, for example, by depression 414 in the rigid base 406 and sunken portion 424 in the film 408 (FIG. 4). The fluid flow path may also include one or more seals, such as frangible seal 413. Frangible seal 413 may be similar to any of the frangible seals discussed above.

Preparation unit 402 may also include a waste chamber 432 for accumulating the fluid sample after the fluid sample passes through fluid analysis chip 404. For example, fluid sample returning to the preparation unit 402 from fluid analysis chip 404 may re-enter the preparation unit 402 via a preparation unit fluid inlet 440. From inlet 440, the fluid sample may flow to waste chamber 432 via a second flow path, the second flow path including at least one fluid conduit 438. The fluid conduit 438 may be formed where the flexible film 408 extends over one or more grooves formed in the top surface of the rigid base portion 406. The fluid conduit 438 may carry the fluid sample entering preparation unit 402 via the inlet 440 to the waste chamber 432. Fluid flow through the fluid conduit 415, the fluid analysis chip 404, and the fluid conduit 438 may be accomplished by drawing a vacuum at waste chamber 432, as discussed above.

FIG. 7 provides a diagrammatic top view illustration of a cartridge 400, including a preparation unit 402 and a fluid analysis chip 404, according to presently disclosed embodiments. In one operational path, a fluid to be analyzed may be provided by sampler 200 after insertion into preparation unit 402. The fluid to be analyzed may be provided to reservoir 410 where it can be mixed with a pre-loaded fluid, such as an aqueous solution of a high molecular weight polymer to form a sample fluid, including a suspension including the fluid to be analyzed mixed with the pre-loaded fluid. Once mixed, a sufficient pressure may be applied to the film covering reservoir 412 to burst frangible seal 413. Upon opening of frangible seal 413, the sample fluid can flow into buffer compartment 414 and then into fluid conduit 430. The sample fluid travels along fluid conduit 430 and exits the preparation unit 402 at preparation unit fluid outlet 436. The sample fluid then travels through fluid analysis chip 404 and re-enters the preparation unit 402 at the preparation unit fluid inlet 440. The sample fluid then travels through fluid conduit 438 and into waste chamber 432.

As noted above, a reader can analyze contents (e.g., cells, particles, target analytes/molecules, etc.) flowing in the sample fluid along a channel or chamber 405 of the analysis chip 404. In some embodiments, the sample fluid contains cells that become focused to the center of flow in the channel based on the viscoelastic properties of the sample fluid (provided by the high molecular weight polymer) in conjunction with the geometry of the channel. This focusing facilitates optical detection of the flowing particles or cells. In this case the particles or cells are counted and differentiated, and their concentration in the original fluid to be analyzed is calculated. In order to be able to deduce the concentration, the depth of the channel must be taken into account according to the following expression:

C=N/(A*h)*R

where “C” is the concentration of cells in the original fluid to be analyzed. “N” is the number of cells counted in the field of view of the reader camera. “A” is the area of the field of view, “h” is the height/depth of the channel, and “R” is the dilution ratio of the fluid to be analyzed in liquid reagents. According to this expression, a variation in height (h) of the channel 405 can directly affect the concentration accuracy.

With reference to FIG. 7, a method of using disposable cartridge 400 will be described. In some embodiments, cartridge 400 may be used in a complete blood count (CBC) where blood cells are differentiated and counted and the hemoglobin content is measured. The CBC test is one of the most common tests performed and having it performed at the Point Of Care, which the use of cartridge 400 may allow, has great value. In cartridge 400, reservoir 410 may be used to store liquid reagents suitable for RBC, platelets, and Leukocytes counting, while the other two chambers 416 and 418 may contain an immunoassay buffer and a plurality of particles, respectively, wherein each particle comprises an antibody that is specific to a target analyte in the fluid sample. As such, the liquid reagents in reservoir 410 may include high molecular weight polymers to facilitate viscoelastic focusing of cells. Thus, reservoir 410 and, separately, reservoirs 416 and 418 represent two different preparation paths within preparation unit 402. Blood is automatically injected from the capillaries of sampler 200 into reservoir 410 and/or reservoir 416 during the insertion of the cartridge into the reader unit. This is achieved by a plunger which pushes the plug to the end of the capillary dispelling the blood into the respective reservoirs. During the insertion the capillaries of the sampler 200 slide through O-rings that seals around the capillaries prior to breaching of the seal in the respective reservoir inlets.

The liquid reagents stored within reservoir 410 may include viscoelastic properties to promote viscoelastic focusing during the flow of cells through channel 405 of the analysis chip 404. For example, cell counting may be performed by means of acquiring images of towing cells (flowing through channel 405 of the analysis chip 404) by a camera or by probing by a focused light beam/laser beam as done in a cytometer. In order to allow reliable counting, the cells may be brought into a focal place of the analyzing optics. Hence, the cells may be aligned in a single plane, e.g., by viscoelastic focusing. The method is based on suspending cells in a focusing medium of certain viscoelastic properties causing the cells suspended therein to align into a single plane if being flowed in a channel of a certain geometry (e.g., having a length of greater than 100 microns and at least one cross-sectional dimension less than 100 microns, e.g., between 5 microns and 100 microns).

Accordingly, the fluid sample (i.e., whole blood) is mixed with the reagents in the respective reservoirs, and once the suspension of the fluid to be analyzed and the pre-loaded reagents and/or particles have been mixed, a pressure is applied on the reservoirs in order to open corresponding frangible seals and enable the sample fluids from either of the preparation paths to pass out of the reservoirs. In one preparation path, the sample fluid flows through the breached seal, into a fluid conduit, and into the buffer chamber 414. This buffer chamber may be important to the operation of the cartridge, as in some embodiments, it may enable the sample fluid to stabilize and aggregate so that it can properly flow into the fluid analysis chip 404. The film 408 covering the buffer chamber may be formed with a geometry that enables expansion and shrinkage in volume, allowing the fluid to fill the buffer chamber and also to be evacuated. For example, once a vacuum is applied to the system (e.g., via a port 434 connected to the waste chamber 432 (FIG. 4) the sample fluid flows through the fluid analysis chip 404 and enters the waste chamber. The waste chamber may include an outlet including a self-sealing plug that enables air to be sucked out, but blocks fluid from exiting the chamber and contaminating the reader unit. The film 408 covering the waste chamber 432 may be flat in order to avoid collapse such that vacuum may be maintained and the waste chamber may be filled.

### Analysis System

As previously described, the cartridge prepares the fluid sample for analysis via the analysis system. In particular, the cartridge is configured to perform one or more assays on the fluid sample and then flow a volume of fluid sample, undergoing, or having undergone, an assay, through a portion thereof to be subsequently analyzed by the analysis system. The analysis system is generally configured to capture images of the fluid sample as it is flowing through a portion of the cartridge and subsequently analyze the images so as to obtain measurements of one or more target analytes/molecules and/or cells within the fluid sample.

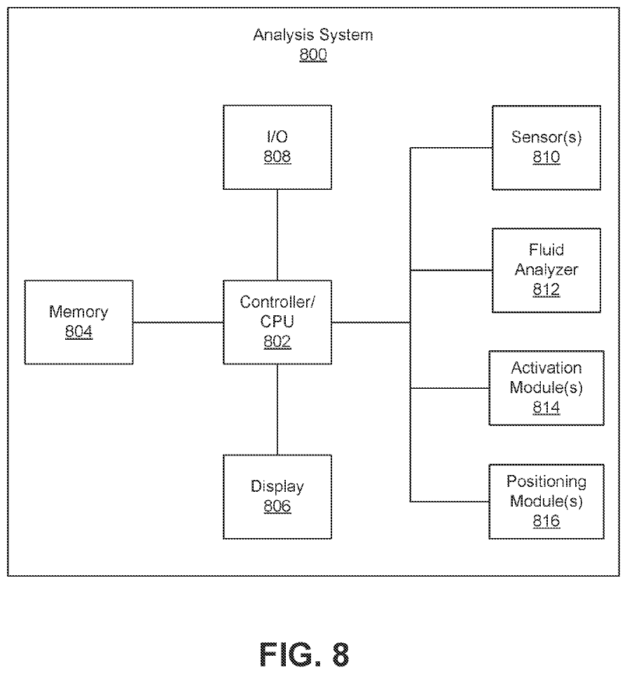

FIG. 8 is a block diagram representation of an analysis system 800 according to some embodiments of the present disclosure. For example, analysis system 800 may include a controller 802 connected either directly or indirectly to various components of analysis system 800. Controller 802 may have access to a memory 804 and may render text and/or images on display 806. In some cases, where display 806 includes a touch sensitive device, controller 802 may receive user commands via the touch-sensitive device associated with display 806. Controller 802 may receive user input and provide various types of output via input-output (I/O) devices 808, which as noted may include various keyboards, point devices, voice recognition modules, etc. Controller 802 may also be connected, for example, via a data bus, to one or more sensors 810, a fluid analyzer 812, a cartridge activation module 814, and a cartridge positioning module 816.

Memory 804 may include any suitable type of data storage device and may include one or more data storage devices either of the same type or of different types. In some cases, memory 804 may include volatile or nonvolatile memory modules. Memory 804 may include, e.g., any combination of RAM, ROM, SRAM, DRAM, PROM, EPROM, EEPROM, magnetic computer readable media, optical computer readable media, flash memory, FPGAs, etc.

Memory 804 may include various types of data and instructions accessible to controller 802. For purposes of this disclosure, references to a controller configured to or programmed to perform certain tasks or functions indicates that memory 804 has been populated with specific data and/or machine executable instructions such that controller 802 can execute those certain tasks or functions by accessing the data and/or instructions and executing one or more instructions included in memory 804. In some cases, memory 804 may include a device separate from controller 802. In other instances, memory 804 may be integrated with controller 802.

Controller 802 may include any suitable logic-based device capable of executing one or more instructions. For example, controller 802 may include one or more digital signal processors, microcontrollers, CPUs, etc. Controller 802 may execute x86 or ARM based instructions or instructions from any other suitable architecture. Controller 802 may include only a single integrated circuit or processing module or may include multiple integrated circuits or processing modules. For example, controller 802 may include one or more applications processors, touch processors, motion control modules, video processors, etc.

Controller 802 may have various functions within system 800. For example, in some embodiments, controller 802 may include electronics and logic for operating or interacting with various components of system 800, including motors, lighting modules, sensors. In certain embodiments, such components may include a pump for aiding in movement of fluids to different parts of a sample holder, pressure gauges, photodiode sensors, LEDs for lighting of a sample fluid, cameras or other types of image acquisition devices for capturing images of a sample fluid. In some embodiments, controller 802 may also interact with or control such components as a touch screen or keyboard and may run various algorithms for implementing processes or functions associated with system 800, including, for example, an autofocus process, image acquisition, processing of acquired sample fluid images, cell counting, cell classification, preparation of a sample fluid in a sample holder, movement of a sample holder to a suitable analysis position within system 800, movement of fluids within a sample holder, among other processes and functions.

For example, cartridge 300, 400 may be introduced into analysis system 800 via receiver 817. In some embodiments, upon insertion of cartridge 300, 400 into system 800, a cartridge holder 818 may retain and otherwise secure cartridge 300, 400 in a desired location within analysis system 800. To prepare a sample included on cartridge 300, 400, activation module 814 may interact with one or more sections of cartridge 300, 400 in order to prepare the fluid sample for analysis. In some embodiments, cartridge activation module 814 may include one or more cams 820 incorporated on a rotating camshaft 822 in order to press (either directly by contacting sections of the cartridge or indirectly by interacting with one or more pistons (or any other suitable structures) that contact the cartridge) sections of the cartridge to prepare, mix, move, distribute, etc. a sample for analysis.

Positioning module 816 may include various components for controlling the position of the prepared sample (e.g., on cartridge 300, 400) relative to analysis components included in fluid analyzer 812. For example, in some embodiments, positioning module 816 may include a motor 824 connected to a stage 826 via a shaft 828. When inserted into analysis system 800, cartridge 300, 400 may be supported either directly or indirectly by stage 826. For example, cartridge holder 818 may include one or more elements to exert a force on cartridge 300, 400 in order to secure cartridge 300, 400 in place on stage 826. Motor 824 may be used to rotate or otherwise move shaft 828 in order to move stage 826. In some cases, stage 826 may be mounted on an inclined rail 830. In such embodiments, control of motor 824 may cause stage 826, and any components coupled to the stage, such as a retained cartridge, for example, to move along inclined rail 830. As a result of the movement along rail 830, stage 826 may simultaneously move both in the X direction and the Z direction relative to fluid analyzer 812.

Fluid analyzer 812 may be mounted to a frame assembly 832, to which motor 824 and inclined rail 830 may also be mounted. Fluid analyzer 812 may include one or more devices for analyzing a fluid sample contained within or included on cartridge 300, 400. In some cases, fluid analyzer 812 may include components for performing flow cytometry based on laser scattering, fluorescence, impedance measurements, etc. Alternatively or additionally, fluid analyzer 812 may include an optical imager, including, for example, lenses, image sensors, and other components suitable for acquiring optical images of the sample. For example, in some embodiments, fluid analyzer 812 may include an optical sensor (such as a CCD, CMOS or photo-multiplier), One or more excitation sources (not shown) may be provided for illuminating a sample fluid to be analyzed with radiation having a wavelength suitable for a selected type of analysis. In some embodiments, the optical sensor may include a camera which acquires images of cells or particles flowing inside an inspection area of cartridge 300, 400. Acquired images may then be processed by controller 802 using suitable software and/or hardware in order to determine, for example, a cell count for one or more cell types present in the sample fluid (e.g., neutrophils, lymphocytes, erythrocytes, etc.). Acquired images, image streams, analysis results, acquired or calculated data, etc. determined or obtained as part of the analysis process may be stored, for example, in memory 804. Detailed descriptions of each of the fluid analyzer 812, activation module 814, and positioning module 816 and the role of each in performing analysis of a fluid sample are included in sections below.

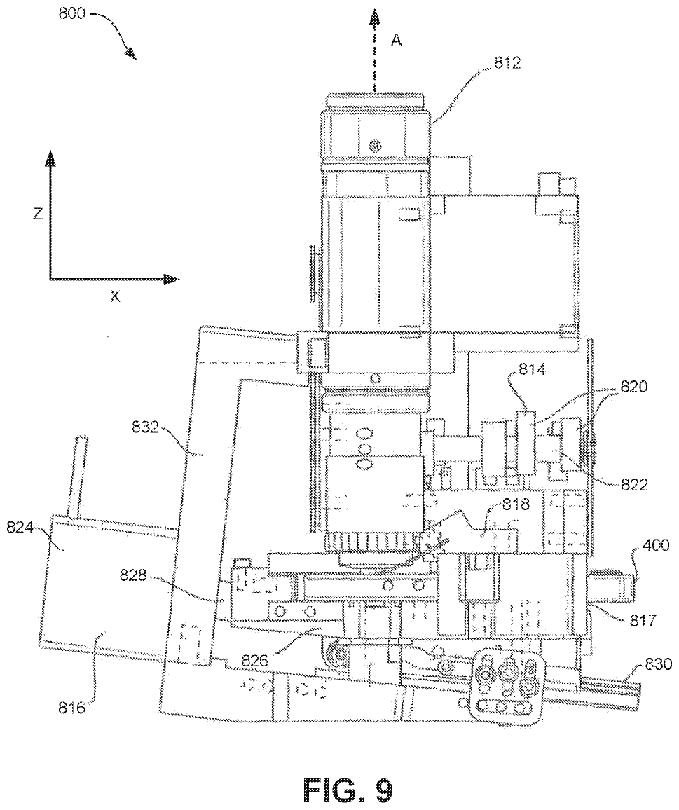

As previously noted with respect to FIG. 8, and as further illustrated in FIG. 9, to prepare a sample included on cartridge 300, 400, activation module 814 may interact with one or more sections of cartridge 300, 400 in order to prepare the fluid sample for analysis. In some embodiments, cartridge activation module 814 may include one or more cams 820 incorporated on a rotating camshaft 822 turned by motor 834, belt 836, and gearing 838 in order to press (either directly by contacting sections of the cartridge or indirectly by interacting with one or more pistons 840 that contact the cartridge) sections of the cartridge to prepare, mix, move, distribute, etc. a fluid sample for analysis. Activation module 814 may include more or few components than the cams, camshaft, motor, belt, pistons, and gearing described.

Cams 820 and/or pistons 840 may be configured to interact with any suitable portions of cartridge 300, 400 in order to prepare a fluid sample for analysis, transport fluid within portions of cartridge 300, 400, open seals, etc. For example, cams and/or pistons 840 may interact with any of the deformable portions, reservoirs, buffer chambers, compartments, fluid channels, etc. described above with respect to any of the various embodiments of cartridge 300, 400. For example, as shown in FIG. 9, rotation of camshaft 822 may cause cams 820 to rotate. By virtue of the various shaped profiles of cams 820, for example, including different shaped lobes radially distributed about camshaft 822, cams 820 may press on pistons 840 to depress pistons 840 at various different times. The cam lobes may be arranged, for example, to cause pistons to alternately press on adjacent fluid reservoirs, as described above, in order to transfer fluid back and forth from one reservoir to another.

The following discussion provides additional details regarding various configurations of activation module 814, including cams 820, camshaft 822, pistons 840, and how activation module 814 interacts with various sections of cartridge 300, 400. For purposes of the disclosure, cams 820 are not limited to any particular structure or configuration. Cams 820 may include modular elements or may include multiple separate components assembled together. Cams 820 may have any suitable thickness and any suitable configuration of lobes. Additionally, any number of camshafts 822 may be employed (e.g., one shaft, two, three, or more). Moreover, motor 834 (or any other driver for activation module 814) may be directly connected to camshaft 822 or may be indirectly attached to camshaft 822 through gearing, belts, etc. (as shown in FIG. 9). The activation module driving mechanism (e.g., motor 834) may include any driving mechanism suitable for causing a desired activation motion (e.g., rotating shafts). The driving mechanism may be configured for driving a rotating mechanism at fixed or variable speeds, and may change speeds and/or rotation direction during operation. Controller 802 may cause motor 834 to rotate camshaft 822 in accordance with a predefined pattern adapted for a particular cartridge configuration.

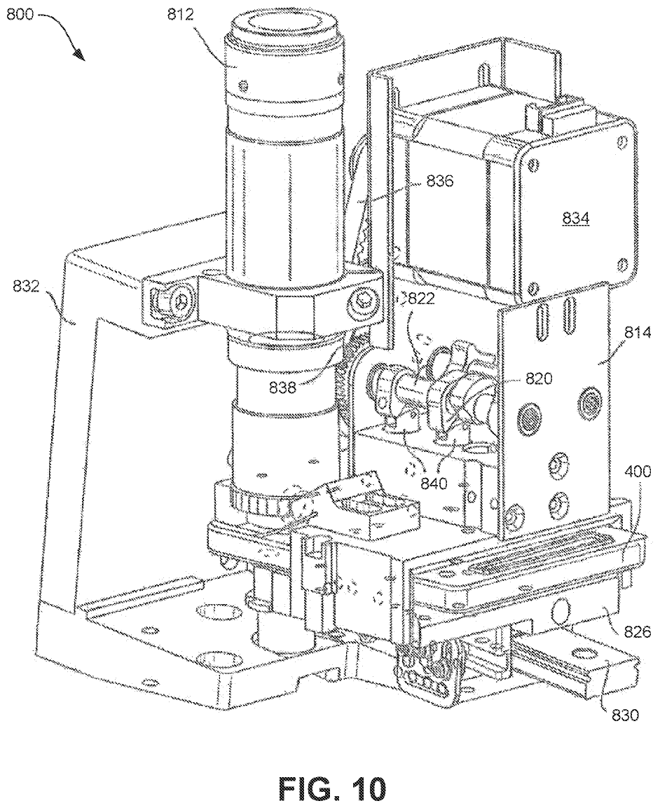

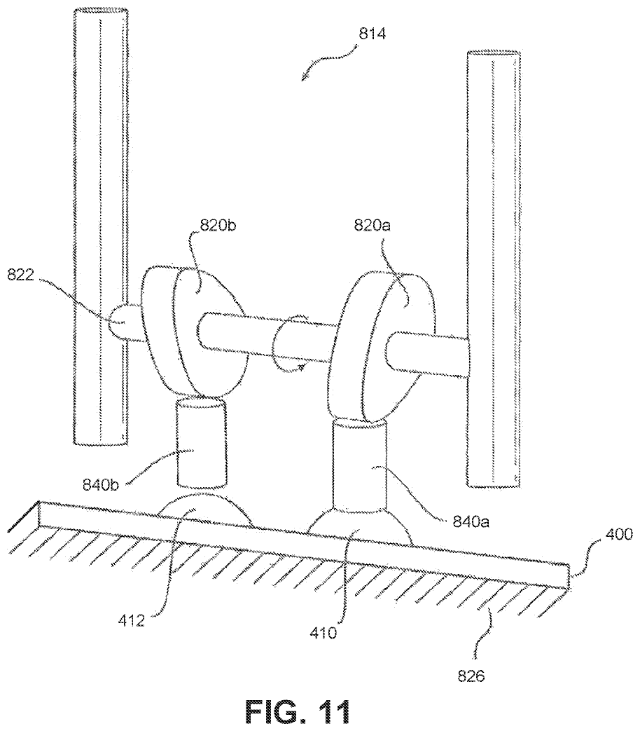

Referring to FIG. 10, driving the camshaft 822 according to a predefined pattern in conjunction with a certain cartridge 300, 400 including deformable elements (e.g., elements that cover or themselves constitute all or part of reservoirs, such as reservoirs 304, 314, 318, and 320 of cartridge 300 or reservoirs 410, 412, 414, 416, and 418 of cartridge 400) results in timed pressing and releasing of the deformable elements in response to movements of cams 820. For example, as shown in FIG. 10, cam 820a has a lobe (or node) contacting piston 840a causing piston 840a to partially depress a deformable element associated with reservoir 410. Cam 820b has a similar profile to cam 820a, but has a different rotational position. As a result, the long lobe of cam 820b has not yet come into contact with piston 840b. When camshaft 822 rotates sufficiently to cause cam 820b to contact piston 840b (which will cause piston 840b to move downward and press on a deformable element associated with reservoir 802), cam 820a will have rotated such that cam 820a no longer contacts piston 840a. Continued rotation of camshaft 822, therefore, will cause periodic and sequential pressing of deformable elements associated with reservoirs 410 and 412, such that fluid may flow back and forth between reservoirs 410 and 412 as cams 820a and 820b rotate through repeated cycles. Of course, the arrangement shown in FIG. 10 is an example only. More or fewer cams may be included. More or few pistons (or no pistons) may be included, and activation module 814 may be configured to depress or interact with any number of different structures associated with cartridge 300, 400. Additionally, cams 820 may have any suitable profile. In some embodiments, any of cams 820 may include a single lobe (node), as shown in FIG. 10, in other embodiments, however, any of cams 820 may include multiple lobes (nodes) positioned at different radial locations. Multiple cams 820 within activation module 814 may be configured with the same or similar width. In other embodiments, cams 820 may include different widths.

In addition to causing pressure to be applied to deformable elements associated with reservoirs 410 and 412, cams 820a and 820b may be configured to cause pressure to other areas of cartridge 300, 400. For example, in some embodiments, the cams may cause pressure to be applied to one or more fluid conduits associated with cartridge 300, 400. Under such pressure, such fluid conduits may pinch shut in order to reduce or prevent the flow of fluid between two or more regions of cartridge 300, 400.

Pressure diagrams can be associated with cams based on their node configurations, Such diagrams may reflect changes in pressure on a deformable element imparted by the nodes of a cam as it rotates. As noted above, any of cams 820 included in activation module 814 may be configured with any desired cam profile (e.g., lobe shape, lobe number, lobe amplitude, cam width, etc.) to provide a desired pressure profile at one or more particular locations of cartridge 300, 400 (e.g., at the deformable members associated with any of the reservoirs of cartridges 300 or 400).

In addition to rotating a cam 820 through a full 360-degree cycle in order to apply pressure to a deformable element of cartridge 300, 400, camshaft 822 may be rotated over a more limited angular range. For example, in some embodiments, a desired pressure profile may be obtained by rotating camshaft 822 and, therefore, earn 820 forward and backward over a portion of the full 8280-degree range (e.g., over a ±10-degree range; a ±20 degree range; or larger or smaller range).

As noted above and referring back to FIG. 9, system 800 may also include a positioning module 816. Positioning module 816 may include various components for controlling the position of the prepared fluid sample (e.g., on cartridge 300, 400) relative to analysis components included in fluid analyzer 812. For example, in some embodiments, positioning module 816 may include a motor 824 (or other suitable type of actuator) connected to a stage 826 via a shaft 828. Motor 824 may be used to rotate or otherwise move shaft 828 in order to move stage 826, on which cartridge 300, 400 may be retained during analysis. Stage 826 may be mounted on an inclined rail 830, such that movement of shaft 828 (which may extend parallel to inclined rail 830) may either pull stage 826 up inclined rail 830 or push stage 826 down inclined rail 830. As a result of the movement along rail 830, stage 826 may simultaneously move both in the X direction and the Z direction relative to fluid analyzer 812. As shown in FIG. 9, the X axis extends in a direction substantially orthogonal to an analysis axis A of fluid analyzer 812, and the Z axis extends in a direction substantially parallel to the analysis axis of fluid analyzer 812. In some cases, e.g., where fluid analyzer 812 includes a imaging device such as a camera, analysis axis A may correspond to an optical axis of the imaging device. Positioning module 816 may include more or fewer components than the motor and shaft described herein for moving stage 826.

Movement of stage 826 along inclined rail 830 may cause a corresponding movement of cartridge 300, 400 residing on or retaining against stage 826. In some embodiments, stage 826 may be configured with a top surface (or a sample supporting surface) that is substantially perpendicular to analysis axis A of fluid analyzer 812 (and the Z direction) and substantially parallel to the X direction (see FIG. 9). Thus, in some embodiments, when cartridge 300, 400 is placed on stage 826, cartridge 300, 400 may be arranged such that an analysis region of cartridge 300, 400 (e.g., channel 310 of cartridge 300 or channel 405 of cartridge 400) extends along the X direction and perpendicular to analysis axis A.

As shown in FIG. 9, inclined rail 830 may be included relative to the X direction. Thus, movement of stage 826 along inclined rail 830 may cause translation of cartridge 300, 400 in both the X and Z directions relative to analysis module 812. In other words, movement of stage 826 along inclined rail 830 may result in a first component of motion for stage 826/cartridge 300, 400 in the X direction (perpendicular to analysis axis A) and a second component of motion for stage 826/cartridge 300, 400 in the Z direction (parallel to analysis axis A). As a result of the motion in the Z direction parallel to analysis axis A, at least a portion of the prepared sample on cartridge 300, 400 may be brought into focus relative to analysis components of fluid analyzer 812. In embodiments where fluid analyzer 812 includes one or more imagers, such focus may include optical focus.

Any suitable type of movement mechanism may be employed in positioning module 816 to move stage 826 along inclined rail 830. In some embodiments, positioning module 816 may include a motor 824. In some cases, motor 824 may include a stepper motor, which may offer the benefits of precision and repeatability. Other types of movement devices may be used, such as servo motors. DC motor encoders, etc. As noted, motor 824 may be coupled to a shaft 828 (either directly or indirectly through one or more coupling components), which, in turn, may be coupled to stage 826 (either directly or indirectly through one or more coupling components). In some embodiments, shaft 828 may interface with stage 826, for example, via threads or a threaded component. In such embodiments, motor 824 may cause shaft 828 to turn through a desired angle of rotation in order to cause a desired amount of translation of stage 826 along inclined rail 830 (via threads on shaft 828 interacting with corresponding threads included in stage 826 or a component associated with stage 826, for example).

Such an arrangement may offer the benefit of providing precision control over sample focusing without requiring similarly precise motors. For example, in an embodiment where a motor or other type of actuator moves a sample directly along the optical axis of an imager to focus the sample relative to the imager, the precision in the focus adjustment may depend on the precision offered by the motor or actuator. And, in applications where micron or sub-micron resolution may be desirable, motors or actuators providing the required level of precision may be costly.

In the presently disclosed embodiments, however, micron or sub-micron resolution may be achieved with motors or actuators that otherwise would not be capable of providing such resolution if configured to move a sample directly along an optical or analysis axis of the analysis module 812. For example, using the disclosed inclined rail arrangement, a translation of stage 826 along inclined rail 830 sufficient to cause R mm of horizontal movement (along the X direction, as shown in FIG. 9) will induce an R×S mm movement in the Z direction (FIG. 9) (where R is horizontal movement in the X direction and S is the slope ratio associated with the inclined rail). For example, in a case where linear rail 830 is configured with a 1/10 slope ratio (S), a translation of stage 826 along inclined rail 830 sufficient to cause 6 microns of horizontal movement (R) in the X direction will result in 0.6 microns of movement in the Z direction. Thus, by leveraging the slope of the inclined rail, the effective vertical focusing precision may be increased significantly (e.g., by a factor of 2, 5, 10, or even higher) over the precision of the motor or other actuator.

While the disclosed system may result in movement along the X axis in addition to the movement along the Z axis used for focusing, such horizontal translation may be inconsequential for a wide range of applications. Using the viscoelastic focusing technique described above, cells or particles suspended in a viscoelastic medium and flowing through a channel of the fluid analysis chip or section of cartridge (e.g., having a length of greater than 100 microns and at least one cross-sectional dimension less than 100 microns, e.g., between 5 microns and 100 microns) may become physically focused or aligned into a single plane. Arrangement of the flowing cells into a single plane may facilitate acquisition of images of the flowing cells by a camera associated with analysis module 812, for example. Such images may be analyzed for performing cell counts.

Flowing the particles or cells to be analyzed along a channel may also facilitate the use of the focusing arrangement described above. For example, because the flowing cells may be physically focused in a plane that extends along channel of the analysis section of analysis chip, analysis may be performed of the cells at any location along a length of the channel where the flowing cells are suitably arranged. For example, in some embodiments, the channel may be about 1 mm wide, 40 microns deep, and 20 mm long (of course, any other suitable dimensions could be used, especially if they allow for a viscoelastic focusing effect). After entering the channel, the cells or particles may align (by viscoelastic focusing, for example) within a short distance of entering the channel. For example, in some embodiments, the physical focusing of the cells or particles may occur within 5 mm or less or 3 mm or less from an inlet to the channel, and they may remain focused as they flow over the remaining length of the channel. The focused cells may align in a plane approximately microns above the bottom of the channel (channel 310 or 405) where the channel has a depth of 40 microns. In order to inspect the cells or particles, analysis module 812 may be positioned anywhere along the inspection region, such as the channel, where the cells or particles to be analyzed exhibit an arrangement suitable for analysis. In the viscoelastic focusing example described, analysis module 812 including, e.g., a camera or other type of imager may be positioned anywhere along channel (channel 310 or 405) such that the field of view of the camera or imager overlaps with an area where the cells or particles to be analyzed are viscoelastically focused into a single plane. For example, assuming a field of view of about 0.3 mm×0.3 mm, images of the viscoelastically focused cells or particles may be acquired anywhere along the channel where the cells or particles are focused. This may include a region anywhere within the 1 mm width of the channel and anywhere from about 3 mm downstream of the channel inlet to the channel outlet, which in the example described above is about 20 mm from the inlet. Because the cells or particles may be flowing, collecting images at different locations along the channel may be inconsequential, as the cells captured from image to image would be changing anyway as a result of the flow.

This flexibility in locating a suitable analysis site is compatible with the focusing system described above, which may include at least some horizontal (X direction) translation along with movements for focusing in the vertical direction (Z). In some cases, the horizontal direction of travel of the stage 826 along inclined rail 830 may be aligned with a channel or other inspection area included on cartridge 300, 400. Thus, as stage 826 translates along inclined rail 830, analysis module 812 may follow the path of the channel or other inspection area. In a particular example, as stage 826 and cartridge 300, 400 move along inclined rail 830 relative to analysis module 812, images of a field of view of 0.3 mm×0.3 mm may be taken over a 5 min length of channel having a width of 1 mm. Assuming a 1/10 slope factor ratio for inclined rail 830, a 5 mm image capture zone along channel may allow for up to 500 microns of Z movement, which may be more than sufficient to enable optical focusing at any location over the entire channel depth (e.g., of about 30 to 40 microns) or substantially beyond.

The presently disclosed embodiments may also include an autofocus function. For example, where analyzer module 812 includes a camera or imager, positioning module 816 may be controlled by controller 802 to automatically move stage 826 as part of an autofocus process for optically focusing imaging components associated with analyzer module 812 upon an area of interest of the fluid to be analyzed. In some embodiments, controller 802 may cause the imaging components of analyzer module 812 to achieve an optical focus coinciding with the location of a viscoelastically focused area of cells within channel (channel 310 or 405).

The autofocus process may proceed according to any suitable process for achieving a desired level of focus relative to the cells or particles to be analyzed. In some embodiments, the autofocus process may proceed by collecting images with analyzer module 812 at a series of positions along the Z axis (by translating stage 826 along inclined rail 830). For example, stage 826 may be translated along inclined rail 830 such that stage moves over a range of 100 microns in the Z direction (or any other suitable distance). Images may be acquired every 2 microns in the Z direction (or at any predetermined distance interval along the rail or at any other suitable interval). The images collected at the various Z locations may be analyzed (with controller 802, for example) to determine a focus level or quality with respect to the cells or particles of interest. In some embodiments, the analysis may include the evaluation of mathematical criteria (e.g., a spatial frequency analysis) that may be indicative of the focus quality at a particular Z position. In some embodiments, higher spatial frequencies may indicate higher focus quality, and lower spatial frequencies may indicate lower focus quality. Based on the scan over the various Z locations/rail locations and analysis of images captured there, the location (e.g., a target location) corresponding to the highest quality observed focus may be determined. To conduct the desired fluid analysis (e.g., cell count, etc.), controller 802 may reposition stage 826 at the target location determined to correspond to the highest observed focus quality.

Additionally, rather than simply moving the stage to the target location initially determined as having the highest observed focus quality and then performing fluid analysis at that location, one or more subsequent scans may be performed. For example, after the first scan over various Z directions, one or mom additional scans may be performed, for example, over increasingly fine movements in the Z direction around the previously determined target location of the highest focus quality, in order to refine the level of focus on the cells or particles of interest. Each subsequent scan may result in a new target location being determined. Such subsequent scans may include Z direction steps of 1.5 microns, 1 micron, 0.5 microns, or less, for example.

In addition to or as an alternative to this iterative autofocusing approach involving a plurality of scans over various Z positions, controller 802 may also calculate a Z position expected to offer the highest focus quality based on a single scan of Z locations. For example, in such a process, the autofocus process may proceed by collecting images with analyzer module 812 at a series of positions along the Z axis (by translating stage 826 along inclined rail 830).

The images collected at the various Z locations/rail locations may be analyzed (with controller 802, for example) to determine a focus level or quality with respect to the cells or particles of interest. The focus quality levels at the various Z locations and/or rail locations may be used to predict the Z location and/or rail location of the highest quality of focus. For example, controller 802 may extrapolate a highest focus quality Z location/rail location (e.g., the target location) based on the observed focus quality values, may use curve fitting techniques, or any other suitable type of calculation to predict the Z location expected to offer the highest focus quality. Once this target location is determined controller 802 may reposition stage 826 along inclined rail 830 such that the stage is positioned at the calculated target location.

It should be noted that the determined Z location offering the highest focus quality (whether observed or calculated) may or may not correspond to any particular distance between analyzer module 812 and cartridge 300, 400, stage 826, or the cells to be analyzed. Rather, in some cases, the determined Z location offering the highest focus quality may correspond only to a value tracked by controller 802 relative to the operation of positioning system 816. In other words, controller 816 may not determine any actual vertical distance Z between any part of the analyzer module and any part of the cartridge or fluid contained therein. Rather, controller 802 may track the position of motor 824 and use this as the basis for tracking observed focus quality values. As each unique motor position, however, may correspond to a unique Z position of cartridge 300, 400, for example, all references in this disclosure to tracked Z position, determined Z position, etc. should be understood as synonymous with tracking, determining, etc. a motor position or any other quantity controller 802 may use to index the movement of stage 826 along inclined rail 830. For example, motion of motor 824 may result in corresponding motion of stage 826 L along inclined rail 830, such that motor position may enable determination of a position of stage 826 along inclined rail 830. Movement of stage 826 (and, therefore, cartridge 300, 400) in the Z direction as a result of a translation L along the inclined rail 830 may be expressed as Z=L*sin(α). At small angles of inclination, tangent is approximately equal to sin, and, therefore, at small angles, the inclination ratio S is approximately sin(α). Accordingly, at small angles of inclination, Z (the component of motion of the stage/cartridge) in the Z direction parallel to analysis axis A is approximately equal to the translation. L, along the inclined rail multiplied by S, the inclination ratio.

In some embodiments, the analysis may include the evaluation of suitable mathematical criteria (e.g., a spatial frequency analysis) that may be indicative of the focus quality at a particular Z position. In some embodiment, higher spatial frequencies may indicate higher focus quality, and lower spatial frequencies may indicate lower focus quality. Based on the scan over the various Z locations and analysis of images captured there, the location of the highest quality focus may be determined. To conduct the desired fluid analysis (e.g., cell count, etc.), controller 802 may reposition stage 826 at the location determined to correspond to the highest observed focus quality.

The disclosed system may also include an autofocus validation step. For example, as noted above, based on observed focus quality values at various Z positions/rail positions, a target position may be calculated. The calculated target position may correspond to the Z position/rail position expected to provide the highest quality focus. To validate the calculation, controller 802 may position stage 826 at the desired Z location/rail position, collect an image via analysis module 812, analyze the collected image, and determine whether the focus quality is as expected.

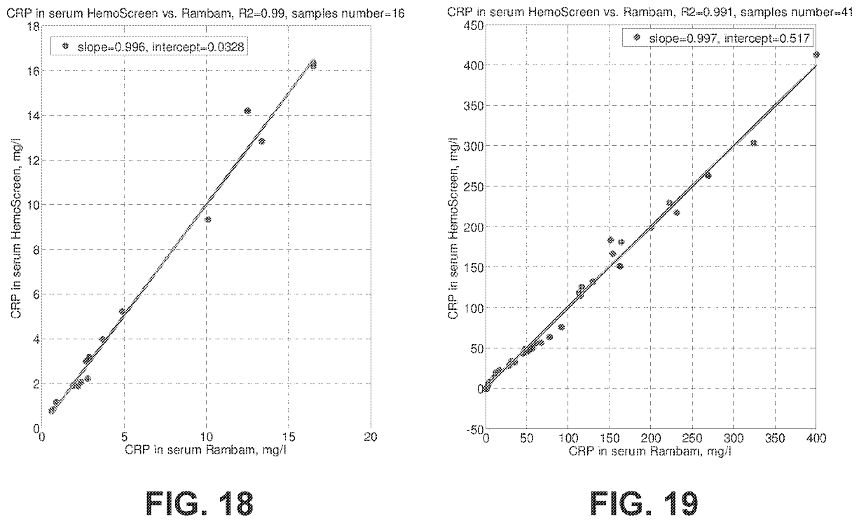

In the disclosed system, certain systems may be associated with one another to provide at least some level of mechanical isolation. For example, as shown in FIGS. 3 and 18, analyzer module 812 may be mounted or coupled to a frame 832, to which motor 824 and inclined rail 830 are also coupled. Stage 826 and activation module 814, however, are not coupled to the frame 832. Rather, both are free to slide together along the inclined rail 830 under the influence of motor 824 and shaft 828, for example.

As a result of this configuration, potential effects on the fluid analysis from the motion of various components in activation module 814 may be reduced or eliminated. For example, by mechanically coupling together stage 826 and activation module 814, the motion of cams 820 and/or pistons 840 may operate to exert a downward force on the cartridge 300, 400, which may be translated to stage 826. Because activation module (including cams 820 and pistons 840) are mounted together with stage 826, however, no force from the motion of cams 820 and/or pistons 840 is transferred to the linear rail 830. This can be beneficial because, any force exerted on the rail could potentially damage the rail or impede the motion of stage 826 along the rail. Moreover, any forces not remaining internal to the cartridge/stage/activation module system could cause relative motion between the cartridge and the analyzer module 812 and, therefore, impact or change the focus of analyzer module 812 relative to the fluid within cartridge 300, 400, which could hinder the fluid analysis.

### Image Analysis

As previously described, the systems and methods of the present disclosure may be used in analyzing a fluid sample having undergone one or more assays wherein analysis of cells and/or target analytes is desired. In particular, the analysis system is configured to capture images of a fluid sample as it is flowing through a channel or chamber of an analysis section/chip of a cartridge and subsequently analyze the images so as to obtain measurements of one or more target analytes/molecules and/or cells within the fluid sample.

In the embodiments described herein, the fluid sample is a whole blood sample, and the cartridge is used in preparing and performing multiple assays on the whole blood sample. For example, a whole blood sample may be loaded into the cartridge, without having to first be separated into a sample of blood serum, and undergo two different assays, including a complete blood count (CBC) assay to obtain CBC measurements, including a hematocrit (Hct) measurement, and a particle-based immunoassay to obtain a concentration measurement of a target analyte from the immunoassay, such as a c-reactive protein (CRP) measurement. An analysis system consistent with the present disclosure (such as system 800), is configured to capture, via magnifying optics and a camera, a plurality of images of a fluid sample as it flows through a channel (a translucent measurement chamber) of the cartridge, analyze the images, and obtain cell count measurements and a concentration of a target analyte based on the analysis.

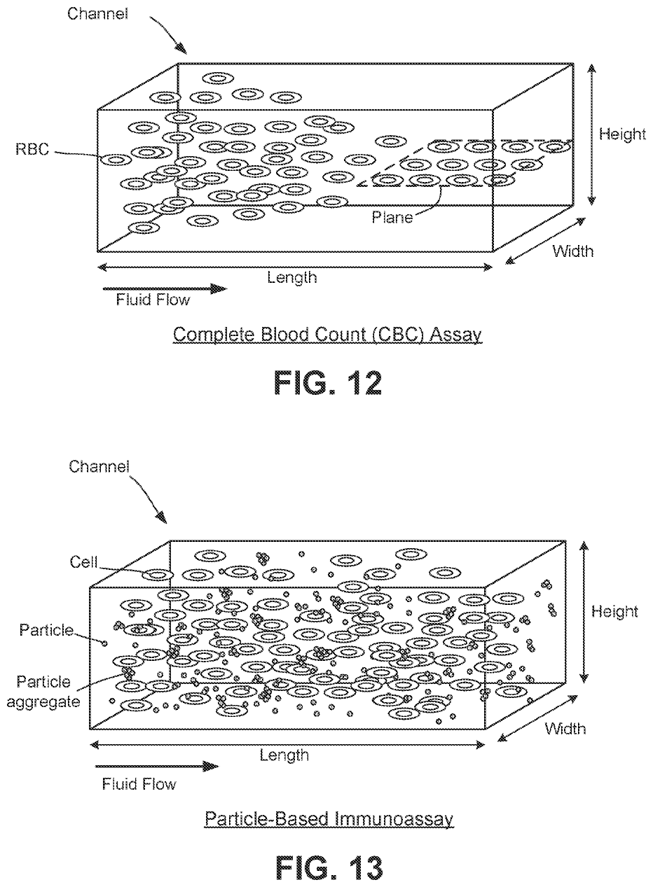

FIG. 12 is a schematic, perspective view of a section of a channel of a cartridge with suspended cells flowing therein as part of a complete blood count (CBC) assay, according to some embodiments of the present disclosure. As shown, the channel has a length, a horizontal dimension (width), and a vertical dimension (height), as indicated by the respective double-arrows. The channel may further be capped by a cover having an interface surface with substrate top surface. In some embodiments, horizontal dimension is in the order of magnitude of 100 micrometers and vertical dimension is in the order of magnitude of 10 micrometers. As previously described, a fluid sample (having undergone, or currently undergoing an assay) flows into and through the channel of the analysis section (or analysis chip) of the cartridge and the analysis system is configured to analyze the fluid as it is flowing. As illustrated, the fluid sample may be a prepared whole blood sample, including cells suspended and flowing through the channel. The analysis system is configured to capture a plurality of images of the fluid as it is flowing through the channel and perform a complete blood count (CBC) based on analysis of the images.

For example, cell counting may be performed by means of acquiring images of flowing cells by a camera or by probing by a focused light beam/laser beam as done in a cytometer. In order to allow reliable counting, the cells may be brought into a focal place of the analyzing optics. Hence, the cells may be aligned in a single plane, e.g., by viscoelastic focusing. The method is based on suspending cells in a focusing medium of certain viscoelastic properties causing the cells suspended therein to align into a single plane if being flowed in a microchannel of a certain geometry (e.g., having a length of greater than 100 microns and at least one cross-sectional dimension less than 100 microns, e.g., between 5 microns and 100 microns). For example, a whole blood sample may be mixed, in reservoirs of the cartridge, with a focusing medium with added surfactants. The focusing medium may include a buffer containing, for example, soluble high molecular weight polymers. The buffer may include any isotonic buffer suitable for managing living cells, including, for example, Phosphate Buffered Saline (PBS). Examples of soluble polymers suitable for providing the blood sample with viscoelastic properties include polyacrylamide (PAA), polyethylene glycol (PEG). Propylene Glycol, etc. The surfactants added to a focusing media may act as sphering agents that may cause the shape of red blood cells to change from biconcave discs into spheres, which may facilitate acquisition of higher quality images of the cells. Examples of surfactants include SDS (Sodium Dodecyl Sylphate) and DDAPS (dodecyl dimethylammonio propanesulfonate). The composition of the focusing medium is disclosed in at least in PCT Publication No. WO2008/149365 entitled “Systems and Methods for Focusing Particles”, which is incorporated herein by reference.

For example, RBC particles suspended in a viscoelastic fluid (not shown) enter at entrance end of the channel and flow downstream in a direction indicated by an arrow. As RBCs enter the microchannel they are still disordered but as RBCs flow downstream they tend to align into a two dimensional array as can be seen about a region in the microchannel indicated by bracket.

It should be noted, that once the RBCs are aligned in an array in a fluid flowing in channel, the flow may be halted or stopped such as by reducing or eliminating the pressure gradient, and the RBCs will remain in an array (‘frozen’) subject to the buoyancy in the viscoelastic fluid and gravity effects. Since the viscoelastic fluid is viscous, in many cases even if the RBCs eventually sink to the microchannel bottom, the sinking is sufficiently slow to allow viewing and analysis of the image in an array as described below. Optionally, the RBCs are fixed (or practically fixed) in place in a motionless fluid by methods such as enlarging the viscosity of the viscoelastic fluid, for example, by cooling (e.g. Peltier effect), or by diffusion of an a suitable chemical agent. Optionally or alternatively, in case the RBCs have electric dipole or are capable to attain induced dipole, the RBCs then can be fixed by applying a suitable electric field. Images of the flowing fluid sample are taken and analyzed to determine cell count or other characteristics of the cells.

FIG. 13 is a schematic, perspective view of a section of a channel of the cartridge with suspended cells and particles flowing therein as part of a particle-based immunoassay, according to some embodiments of the present disclosure. The analysis instrument may utilize a specialized algorithm during the image analysis process, in which cells within a fluid sample (i.e., red blood cells, white blood cells, bacterial cells, etc.) may be classified and differentiated from a plurality of particles. Accordingly, the analysis instrument may be configured to differentiate between intact cells and the particles within the suspension of fluid sample. For example, as shown in FIG. 13, a fluid sample, having undergone, or currently undergoing, a particle-based immunoassay, flows through a channel of an analysis section (analysis chip) of a cartridge. The fluid sample may include whole blood, for example, and the particles may include an antibody that is specific to a target analyte (e.g., c-reactive protein), such that the c-reactive protein in a fluid sample will bind to the particle, via the antibody, to form one or more aggregates. The suspension of cells and particles flows through a translucent measurement chamber where images of the flowing particles are captured via magnifying optics and a camera, upon which the images are analyzed. In particular, the analysis system is configured to analyze dynamics of aggregation of the particles within the flowing fluid sample to determine a concentration of a target analyte in the fluid sample.

It should further be noted that, in some embodiments, the fluid sample may undergo a lysing procedure, which may be useful in measuring intra-cellular proteins, such as HbA1C. Accordingly, the fluid sample flowing through the channel of the cartridge may include lysed cells (i.e., non-intact cells), including cellular debris, as well as particles bound to one or more target analytes, such as intra-cellular proteins. Accordingly, in some embodiments, a suspension of cellular debris and particles (which are bound to a target analyte, such as an intra-cellular protein) flows through a translucent measurement chamber where images of the flowing particles are captured via magnifying optics and a camera, upon which the images are analyzed. The analysis system is configured to perform image analysis on the flowing fluid sample, which may include obtaining a plurality of different images, wherein the system is configured to exclude cellular debris within any given image, while analyzing dynamics of aggregation of the particles within the flowing fluid sample to determine a concentration of the intra-cellular protein in the fluid sample over a period of time.

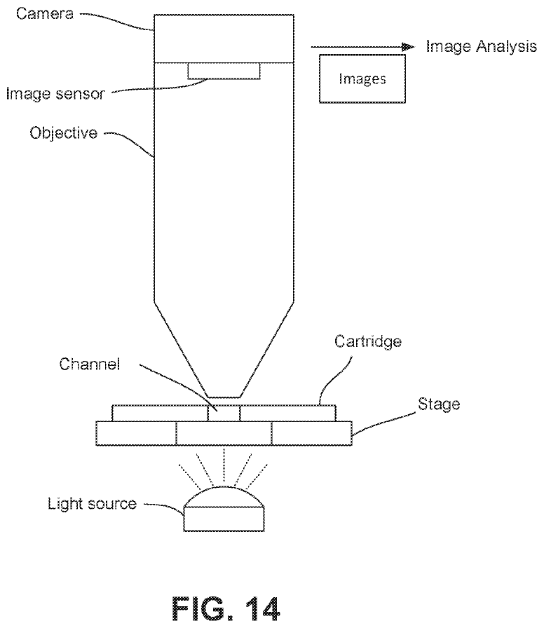

FIG. 14 is a schematic illustration of a fluid analysis system capturing images of a fluid sample flowing through the channel of the cartridge, according to exemplary embodiments of the present disclosure. As illustrated, the system may include an illumination source, an optical objective, and a camera. The illumination source provides light that passes through the stage of the analysis system and through the cartridge and at least partly through the contents of the fluid flowing through the channel of the cartridge, and objective optically projects the image on an image sensor (e.g. CMOS or CCD) in camera. The camera captures the image off of the image sensor and provides the image, possibly after a transformation and/or pre-processing, to a display for a visual observation and further transfers the image for subsequent image processing for analysis to provide one or more qualitative or quantitative results.

The illumination source provides light in a suitable color to produce an image of good quality, for example, an image having best attainable or sufficient or reasonable sharp and/or distinct and/or contrasted shapes of the particles. In some embodiments, the light is monochromatic and optionally the color is selected from a pre-set group or according to the capabilities of illumination source. Optionally or alternatively, the color is variably set, such as according to the nature and/or color of the particles. In some embodiments, the light is polarized or provided as dark field or other illumination techniques used in the microscopy art.

In some embodiments, the illumination source illuminates the cartridge on the stage from above and the objective projects an image onto the sensor according to light reflected from the particles in the microchannel. Optionally or alternatively, two or more light sources may be included which illuminate from below and/or above of the cartridge in colors and intensities to increase or maximize the quality of the cells and/or particles image (such as in terms of sharpness, contrast, etc.).

The analysis system is configured to analyze the images to determine characteristics of components (i.e., cell count, cell type, particle aggregation, etc.) based on extraction of information embedded in the images. For example, such methods may include one or more of the following techniques: segmentation or blob analysis; isolating and/or extracting and determining the shapes or morphology of the cells and/or particles (e.g., round, elongated, branching, fiber-like, fibrous, helical, or looping); and determining features such as convexity or eccentricity. In some embodiments, based on shapes of regions of the image and/or on extracted particles shapes the program provides calculations or estimations of the sizes and/or volumes and/or density and/or concentration of the particles.

In some embodiments, the program provides values based on one or more derivations and/or manipulations of extracted or determined features (e.g. number and size of cells and/or particles). Optionally the derivation comprises employing experimental or assumed values such as known in the art and/or derived from a calibration procedure or otherwise obtained. Optionally, based on the shape or size or concentration of the cells and/or particles or other determined data, the system provides a value or as indication or a suggestion of at least one of a biological or clinical significance, for example, inference or indication of possible or plausible physical or physiological or pathological condition.

With regard to the particle-based immunoassay, the images of the gradually aggregating particles are analyzed on the fly using image processing algorithms to determine a concentration of the target analyte in the fluid sample. For example, dynamics of formation of the one or more aggregates is analyzed in each image, such that the dynamics of formation can be analyzed over a period of time. The dynamics of formation of aggregation may include, but is not limited to, a rate of formation of the one or more aggregates and a size of the one or more aggregates. The analysis instrument is configured to monitor, not only the size and rate of formation of the aggregates, but further monitor one or more characteristics of the particles, such as color, size, and/or shape. The sizes of the particles are monitored as well as their colors or morphology (for multiplexing purposes) and thus the dynamics of aggregation are recorded.

As such, systems and methods of the invention allow for multiple immunoassays to be simultaneously performed on a fluid sample such that different target analytes may be detected and their associated concentrations may be measured, as different particles may have different characteristics, such as color or morphology (e.g., a first set of particles to bind to a first target analyte have a first color and a second set of particles to bind to a second target analyte have a second color). This multiplexing ability is particularly important in the diagnosing of certain infection and disease states, such as bacterial infection, cancer, or heart failure, as the combination of several biomarkers provides much better sensitivity than each one alone. The analysis instrument may utilize a specialized algorithm during the image analysis process, in which cells within a fluid sample (i.e., red blood cells, white blood cells, bacterial cells, etc.) may be classified and differentiated from the plurality of particles. As such, the cells may be classified using machine learning algorithms and differentiated from the particles. In particular, intact cells are excluded by a technique including processing an image of intact cells to produce a background threshold, processing an image of the fluid sample comprising the intact cells and one or more aggregates, and normalizing the image of the fluid sample against the background threshold, thereby excluding intact cells from the image analysis of the fluid sample. From these measurements the concentration of several analytes can be deduced as well as cell concentration.

Accordingly, the analysis instrument may be configured to differentiate between intact cells and the particles within the suspension of fluid sample. As such, the systems and methods of the invention further allow for additional assays to be performed on the fluid sample (e.g., non-immune response assays) so as to obtain measurements related to specific components within the fluid sample (i.e., cell counting and characterization). For example, a whole blood sample may be loaded into the cartridge, without having to first be separated into a sample of blood serum, and undergo two different assays, such as a complete blood count (CBC) assay and an immunoassay. In certain embodiments, constant flow allows measuring a large portion of the suspension and thus higher accuracy and repeatability are achieved. The imaging-based analysis allows monitoring the particles aggregation without cells interference by inspecting the space between the cells, thereby disregarding the cells. The complete size distribution at any given time is attained, which provides more information on the reaction. Finally, image analysis enables multiplexing several assays by using colored beads or differently size or shaped beads.

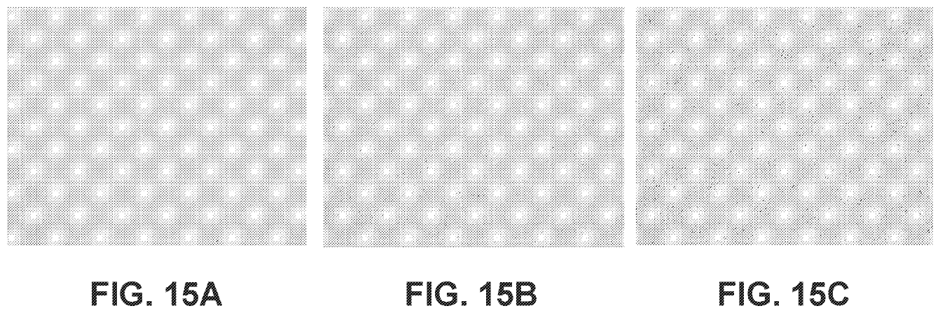

FIGS. 15A, 15B, and 15C are images of aggregation of particles within a fluid sample, undergoing a particle-based immunoassay and absent cells, flowing through a channel of the cartridge, wherein each image is captured at a different respective time period. For example, FIG. 15A is an image captured immediately upon a fluid sample undergoing an immunoassay for CRP, while FIGS. 15B and 15C are images captured after four minutes and twelve minutes have elapsed, respectively, illustrating the increase in aggregation of particles (i.e., increase in the binding of particles to target CRPs).

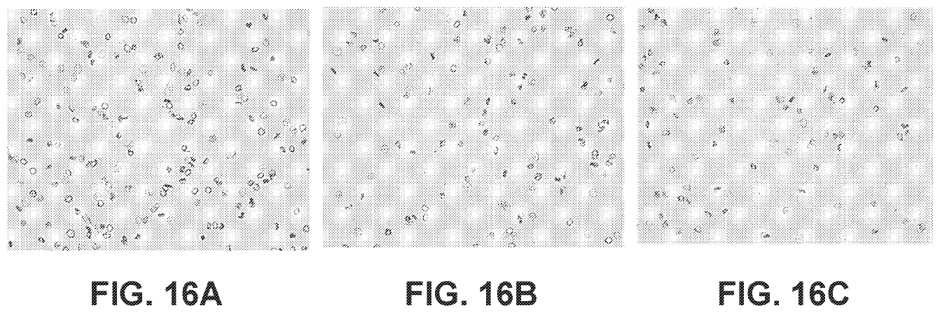

FIGS. 16A, 16B, and 16C are images of aggregation of particles within a fluid sample, undergoing a particle-based immunoassay and including intact cells, flowing through a channel of the cartridge, wherein each image is captured at a different respective time period. FIG. 16A is an image captured immediately upon a fluid sample undergoing an immunoassay for CRP, while FIGS. 16B and 16C are images captured after four minutes and twelve minutes have elapsed, respectively, illustrating the increase in aggregation of particles (i.e., increase in the binding of particles to target CRPs). Again, the analysis system of the present disclosure is configured to differentiate between intact cells and the particles within the suspension of fluid sample when performing image analysis, thereby allowing for a whole blood sample to be used (where conventional systems are unable to differentiate between intact cells and particle aggregation).

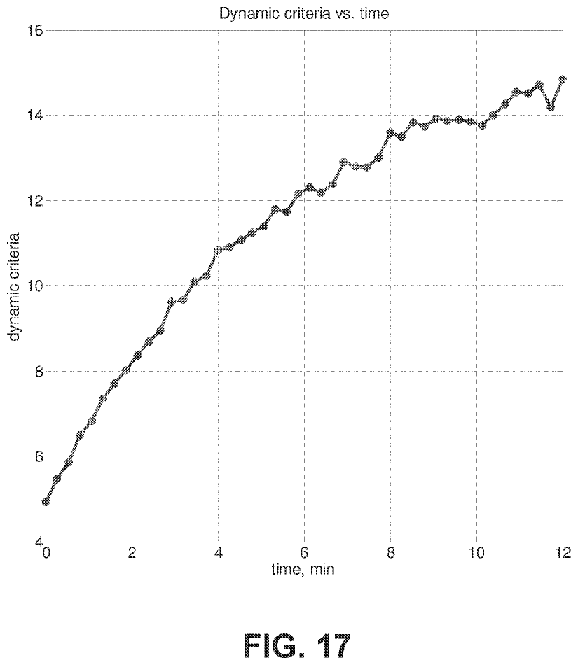

FIG. 17 is a graphical representation illustrating dynamics of aggregation of particles over a period of time. The dynamics of aggregation (e.g., the rate and size of aggregates) is indicative of the molecule concentration. The system of the present disclosure is configured to measure dynamics of aggregation of the particles on the fly and over a period of time, thereby improving accuracy of determination of a concentration of the target analyte/molecule.

FIGS. 18 and 19 are graphical representations illustrating the accuracy of c-reactive protein measurements performed in accordance with the image-based analysis systems and methods of the present disclosure as compared to existing c-reactive protein measurements obtained via existing analysis platforms.

### Exemplary Methods of Fluid Analysis

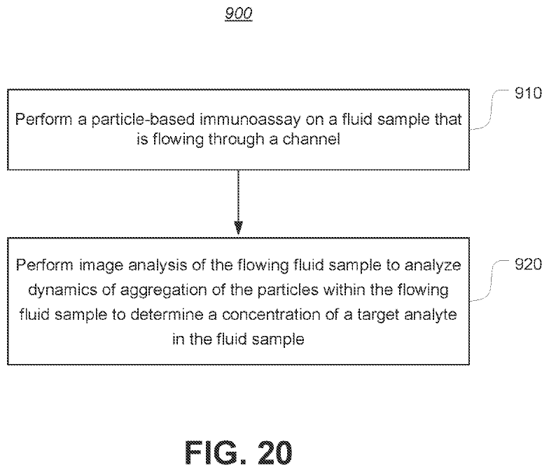

FIG. 20 is a flow diagram illustrating one embodiment of a method 900 for analyzing a fluid sample. The method 900 includes performing a particle-based immunoassay on a fluid sample that is flowing through a channel (operation 910) and performing image analysis of the flowing fluid sample to analyze dynamics of aggregation of the particles within the flowing fluid sample to determine a concentration of a target analyte in the fluid sample (operation 920). The step of performing image analysis may include obtaining a plurality of different images and analyzing the dynamics of formation of the one or more first aggregates in each image. The images may include, for example, a vertical scan along a height of the channel. In particular, a vertical scan along the height of the channel may be required due to the fact that particles may be dispersed across the height of the channel and the number of particles observed in an image may vary with distance from the center of channel. Thus, a series of images along a channel's height may be required in order to realize where the center of the channel is. The dynamics of formation of aggregation may include a rate of formation of the one or more aggregates, a size of the one or more aggregates, and a combination thereof. As such, the determination of a concentration of the target analyte in the fluid sample may be based, at least in part, on a rate of formation of the one or more aggregates, a size of the one or more aggregates, and a combination thereof. The fluid sample may include whole blood and the target may include, but is not limited to, a c-reactive protein (CRP), HbA1C, PCT, BNP, and a combination thereof.

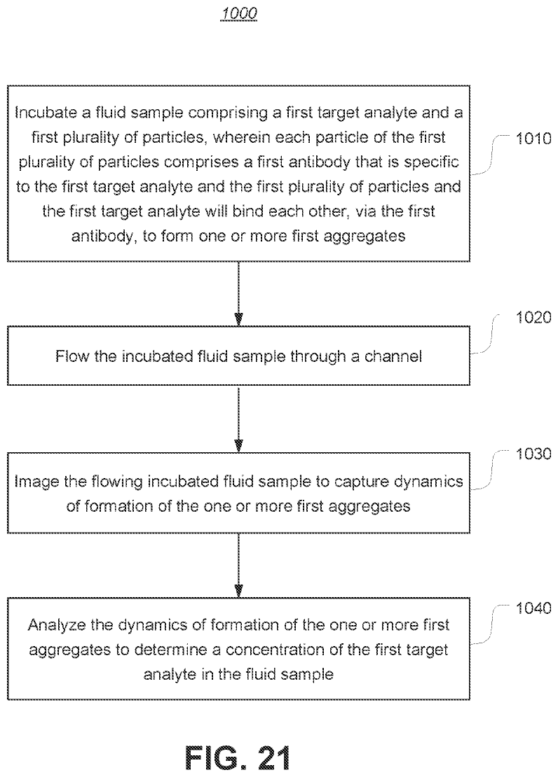

FIG. 21 is a flow diagram illustrating another embodiment of a method 1000 for analyzing a fluid sample. The method 1000 includes incubating a fluid sample comprising a first target analyte and a first plurality of particles (operation 1010). Each particle of the first plurality of particles comprises a first antibody that is specific to the first target analyte and the first plurality of particles and the first target analyte will bind each other, via the first antibody, to form one or more first aggregates. The method 1000 further includes flowing the incubated fluid sample through a channel (operation 1020), imaging the flowing incubated fluid sample to capture dynamics of formation of the one or more first aggregates (operation 1030), and analyzing the dynamics of formation of the one or more first aggregates to determine a concentration of the first target analyte in the fluid sample (operation 1040). In some embodiments, the fluid sample may include a second target analyte such that the incubating step further includes a second plurality of particles, wherein the second plurality of particles comprise an optical characteristic that is different from the first plurality of particles, each particle of the second plurality of particles comprises a second antibody that is specific to the second target analyte, and the second plurality of particles and the second target will bind each other, via the second antibody, to form one or more second aggregates. Accordingly, the method 1000 may further include imaging the flowing incubated fluid sample to capture dynamics of formation of the one or more second aggregates and analyzing the dynamics of formation of the one or more second aggregates to determine a concentration of the second target analyte in the fluid sample. In some embodiments, the fluid sample may include intact cells such that the method 1000 is conducted in the presence of the intact cells. Accordingly, the analyzing step may exclude the intact cells that are present in the imaged fluid sample. The intact cells may be excluded by a technique that includes processing an image of intact cells to produce a background threshold, processing an image of the fluid sample includes the intact cells and one or more first aggregates, and normalizing the image of the fluid sample against the background threshold, thereby excluding intact cells from the analysis of the fluid sample.

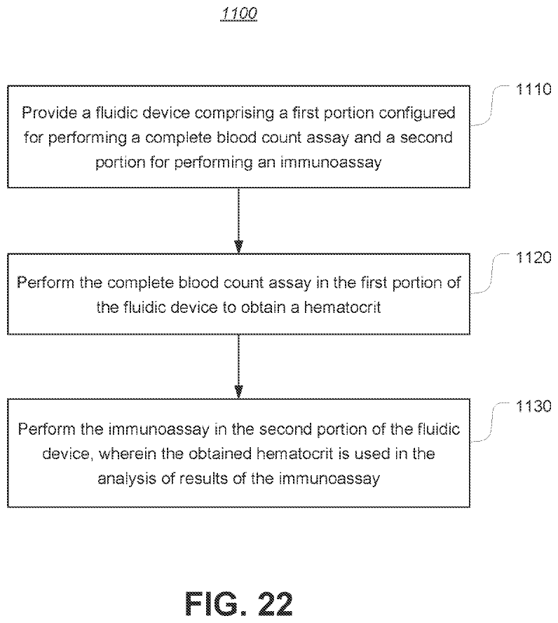

FIG. 22 is a flow diagram illustrating another embodiment of a method 1100 for analyzing a fluid sample. The method 1100 includes providing a fluidic device includes a first portion configured for performing a complete blood count assay and a second portion for performing an immunoassay (operation 1110), performing the complete blood count assay in the first portion of the fluidic device to obtain a hematocrit (operation 1120), and performing the immunoassay in the second portion of the fluidic device, wherein the obtained hematocrit is used in the analysis of results of the immunoassay (operation 1130).

Accordingly, the present invention recognizes the drawbacks of current analysis instruments and provides systems and methods for performing one or more immunoassays on a fluid sample and performing image analysis on the fluid sample as the sample is flowing so as to obtain measurements related to one or more target analytes based on image analysis. Particularly, aspects of the present invention provide system and methods for performing one or more immunoassays using image analysis and flow, as well as an ability to perform assays (such as immunoassays) on a fluidic device (e.g., a disposable cartridge) facilitating use at the point-of-care (POC). The unique combination of image analysis, microfluidics, immunoturbidimetry and innovative fluidic devices (e.g., cartridges) solves the above problems.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of analyzing a fluid sample, the method comprising:
performing a particle-based immunoassay on a fluid sample that is flowing through a channel; and
performing image analysis of the flowing fluid sample to analyze dynamics of aggregation of the particles within the flowing fluid sample to determine a concentration of a target analyte in the fluid sample.

2. The method of claim 1, wherein performing image analysis comprises obtaining a plurality of different images, wherein dynamics of formation of the one or more first aggregates is analyzed in each image.

3. The method of claim 2, wherein performing image analysis comprises obtaining a vertical scan along a height of the channel.

4. The method of claim 1, wherein dynamics of formation of aggregation comprises at least one selected from the group consisting of rate of formation of the one or more aggregates, size of the one or more aggregates, and a combination thereof.

5. The method of claim 1, wherein performing the particle-based immunoassay on the fluid sample further comprises:
providing a first plurality of particles, wherein each particle of the first plurality of particles comprises a first antibody that is specific to a first target analyte and the first plurality of particles and the first target analyte will bind each other, via the first antibody, to form one or more first aggregates; and
providing a second plurality of particles, wherein the second plurality of particles comprise an optical characteristic that is different from the first plurality of particles, each particle of the second plurality of particles comprises a second antibody that is specific to the second target analyte, and the second plurality of particles and the second target will bind each other, via the second antibody, to form one or more second aggregates.

6. The method of claim 5, wherein performing image analysis of the flowing fluid sample further comprises:
imaging the flowing incubated fluid sample to capture dynamics of formation of the one or more first aggregates and one or more second aggregates; and
analyzing the dynamics of formation of the one or more first aggregates and the one or more second aggregates to determine a concentration of the first target analyte in the fluid sample and the second target analyte in the fluid sample.

7. The method of claim 1, wherein the fluid sample comprises intact cells and the method is conducted in the presence of the intact cells.

8. The method of claim 7, wherein the image analysis excludes the intact cells that are present in the imaged fluid sample.

9. The method of claim 8, wherein the intact cells are excluded by a technique comprising:
processing an image of intact cells to produce a background threshold;
processing an image of the fluid sample comprising the intact cells and one or more aggregates; and
normalizing the image of the fluid sample against the background threshold, thereby excluding intact cells from the image analysis of the fluid sample.

10. The method of claim 1, wherein the performing step comprises:
providing a cartridge;
introducing the fluid sample comprising a first target analyte into a reservoir of the cartridge, the reservoir comprising a first reagent;
incubating the fluid sample with the first reagent;
flowing the fluid sample into a second reservoir of the cartridge comprising a first plurality of particles, wherein each particle of the first plurality of particles comprises a first antibody that is specific to the first target analyte and the first plurality of particles and the first target analyte will bind each other, via the first antibody, to form one or more first aggregates;
flowing the fluid sample and first plurality of particles through a channel in the cartridge;
imaging the flowing fluid sample to capture dynamics of formation of the one or more first aggregates; and
analyzing the dynamics of formation of the one or more first aggregates to determine a concentration of the first target analyte in the fluid sample.

11. A method of analyzing a fluid sample, the method comprising:
incubating a fluid sample comprising a first target analyte and a first plurality of particles, wherein each particle of the first plurality of particles comprises a first antibody that is specific to the first target analyte and the first plurality of particles and the first target analyte will bind each other, via the first antibody, to form one or more first aggregates;
flowing the incubated fluid sample through a channel;
imaging the flowing incubated fluid sample to capture dynamics of formation of the one or more first aggregates; and
analyzing the dynamics of formation of the one or more first aggregates to determine a concentration of the first target analyte in the fluid sample.

12. The method of claim 11, wherein imaging comprises obtaining a plurality of different images, wherein dynamics of formation of the one or more first aggregates is analyzed in each image.

13. The method of claim 11, wherein imaging comprises obtaining a vertical scan along a height of the channel.

14. The method of claim 11, wherein dynamics of formation of the one or more aggregates comprises at least one selected from the group consisting of rate of formation of the one or more aggregates, size of the one or more aggregates, and a combination thereof.

15. The method of claim 11, wherein the fluid sample comprises a second target analyte and the incubating step further comprises a second plurality of particles, wherein the second plurality of particles comprise an optical characteristic that is different from the first plurality of particles, each particle of the second plurality of particles comprises a second antibody that is specific to the second target analyte, and the second plurality of particles and the second target will bind each other, via the second antibody, to form one or more second aggregates.

16. The method of claim 15, further comprising:
imaging the flowing incubated fluid sample to capture dynamics of formation of the one or more second aggregates; and
analyzing the dynamics of formation of the one or more second aggregates to determine a concentration of the second target analyte in the fluid sample.

17. The method of claim 11, wherein the fluid sample comprises intact cells and the method is conducted in the presence of the intact cells.

18. The method of claim 17, wherein the analyzing excludes the intact cells that are present in the imaged fluid sample.

19. The method of claim 18, wherein the intact cells are excluded by a technique comprising:
processing an image of intact cells to produce a background threshold;
processing an image of the fluid sample comprising the intact cells and one or more first aggregates; and
normalizing the image of the fluid sample against the background threshold, thereby excluding intact cells from the analysis of the fluid sample.

20. The method of claim 11, wherein the fluid sample is whole blood and the target is selected from the group consisting of a c-reactive protein, HbA1C, PCT, BNP, and a combination thereof.

21. A method for analyzing a fluid sample, the method comprising:
providing a fluidic device comprising a first portion configured for performing a complete blood count assay and a second portion for performing an immunoassay;
performing the complete blood count assay in the first portion of the fluidic device to obtain a hematocrit; and
performing the immunoassay in the second portion of the fluidic device, wherein the obtained hematocrit is used in the analysis of results of the immunoassay.

22. The method of claim 21, wherein the immunoassay is performed using image analysis to analyze dynamics of formation of aggregates in the fluid sample.

23. The method of claim 22, wherein the immunoassay is performed on whole blood comprising intact cells.

24. The method of claim 23, wherein the immunoassay is performed without lysing the intact cells.

25. The method of claim 23, wherein the image analysis excludes the intact cells that are present in the imaged fluid sample.

26. The method of claim 25, wherein the intact cells are excluded by a technique comprising:
processing an image of intact cells to produce a background threshold;
processing an image of the fluid sample comprising the intact cells and one or more aggregates; and
normalizing the image of the fluid sample against the background threshold, thereby excluding intact cells from the image analysis of the fluid sample.

27. The method of claim 22, wherein the immunoassay is performed on a flowing fluid sample.

28. The method of claim 21, wherein the wherein the fluidic device is a cartridge that is configured to be operably coupled to an analytical instrument.

29. The method of claim 28, wherein cartridge is pre-loaded with reagents for each of the complete blood count assay and the immunoassay.

30. The method of claim 21, wherein the immunoassay is performed to determine a concentration of a target analyte in the fluid sample, wherein the target analyte is at least one selected from the group consisting of a c-reactive protein, HbA1C, PCT, BNP, and a combination thereof.

31. A fluid cartridge comprising:
one or more reservoirs comprising a reagent for an immunoassay and a first plurality of particles, wherein each particle of the first plurality of particles comprises a first antibody that is specific to a first target analyte in a fluid sample;
a seal between the one or more reservoirs; and
a first channel operably coupled to the one or more reservoirs to receive and flow fluid from the one or more reservoirs.

32. The fluid cartridge of claim 31, wherein the one or more reservoirs further comprise magnetic particles.

33. The fluid cartridge of claim 31, wherein at least one of the one or more reservoirs comprises a deformable cover that can be deformed into one or more pre-threshold and post-threshold configurations, and the seal is configured to burst only when the deformable cover is in one of the plurality of post-threshold configurations.

34. The fluid cartridge of claim 31, wherein the cartridge further comprises a first reservoir comprising an immunoassay buffer, a second reservoir comprising the first plurality of particles that is fluidically coupled to the first reservoir, and at least a third reservoir associated with an inlet that is different from an inlet to the first reservoir and the second reservoir.

35. The fluid cartridge of claim 34, wherein the third reservoir comprises one or more reagents for performing a complete blood count assay.

36. The fluid cartridge of claim 35, further comprising a second channel operably coupled to the third reservoir to receive and flow fluid from the third reservoir.

37. The fluid cartridge of claim 36, wherein the cartridge is configured such that the first channel and the second channel are coupled to a common junction that is downstream from the first, second, and third reservoirs.

38. The fluid cartridge of claim 37, wherein the cartridge is configured such that when fluid flow through the first channel arrives at the common junction, the fluid flow from the first channel displaces and reverses the fluid flow from the second channel.

39. The fluid cartridge of claim 38, further comprising a third channel coupled to the common junction.

40. The fluid cartridge of claim 39, wherein the cartridge is configured to be operably coupled to an analytical instrument configured to perform image analysis on fluid sample flowing through the third channel.
